(19) United States
(12) Patent Application Publication
(10) Pub. No.: US 2025/0361170 A1
(43) Pub. Date: Nov. 27, 2025

(54) Redrawable glass, light guide element having said glass, and uses thereof

(71) Applicant: SCHOTT AG, Mainz (DE)

(72) Inventor: Bianca SCHREDER, Mainz (DE)

(21) Appl. No.: 19/206,674

(22) Filed: May 13, 2025

(30) Foreign Application Priority Data
May 27, 2024 (DE) 102024114790.3

(51) Int. Cl. C03C 3/068; A61B 1/00; C03C 3/091; C03C 25/106; G02B 6/02

(52) U.S. Cl. CPC C03C 3/068; C03C 3/091; C03C 25/1061; A61B 1/0017; C03C 2203/10; C03C 2203/52; C03C 2217/29; G02B 6/02

(57) ABSTRACT

A redrawable glass, in particular for light guide elements (1) such as glass fibres, is provided. In particular, highly transparent glasses, a method for producing same, and uses thereof. The glasses are preferably used as core glass in a light and/or image guide (1). A light and/or image guide (1) that includes the glass as core glass (2), and a cladding glass (3) is also provided. The use of such a glass in the fields of medical technology, in particular for endoscopic applications, imaging, projection, telecommunications, optical data transmission technology, mobile drive, laser technology and disinfection, and also optical elements or preforms of such optical elements.

This claims priority to German patent application DE 10 2024 114 790.3, filed on May 27, 2024, which is hereby incorporated by reference herein.

The invention relates to redrawable glass, in particular for glass fibres.

### BACKGROUND OF THE INVENTION

Fibre optic light guides are becoming increasingly widespread for the transmission of light in a wide variety of technical and medical fields, e.g. in general industrial technology, lighting and transport technology, the automotive industry, in medical technology such as dental medicine or endoscopy, etc. Because of their good thermal and chemical resistance, use is usually made of fibre optic light guides made of glass, consisting of bundles of individual fibres assembled together. The individual light guide fibres guide the light by total internal reflection. The most widespread light guide fibres are step-index fibres which consist of a core made of core glass, the core glass having a constant refractive index over the cross section thereof. The core glass is surrounded by a cladding made of cladding glass, which has a lower refractive index than the core glass. Total internal reflection occurs at the interface between the core and cladding glass.

The amount of light coupled into such a fibre is proportional to the square of the numerical aperture (NA) of the fibre and the cross-sectional area of the fibre core. The NA corresponds to the sine of the angular range within which light can be accepted by the fibre. The angular range is also referred to as the angular aperture.

### SUMMARY OF THE INVENTION

In addition to the numerical aperture, the attenuation of the light in the fibre is also highly important. Therefore, only core glasses having low attenuation can be used. Because of their high purity, the raw materials for melting such core glasses are extremely expensive, which can lead to substantial costs to manufacture such fibres, or the light and/or image guides produced therefrom. Furthermore, for reasons of environmental protection, toxic constituents such as PbO, CdO, As₂O₃, BeO, HgO, Tl₂O, ThO₂ should no longer be used.

Furthermore, particularly in mobile applications, the reliability of the fibre is important, i.e. the resistance to ageing under thermal cycling stresses between approximately −50° C. and 110° C., resistance to mechanical stresses, in particular vibration resistance, and chemical resistance to environmental influences and cleaning procedures. Weather resistance and the resistance of the core to alkaline solutions are of particularly importance. The density of the fibres is also important, because this has a direct influence on the payload and fuel consumption of an aircraft or car. The density of a step-index fibre is mainly determined by the density of the core glass.

Stepped optical fibres made of multi-component glasses are produced either using the double crucible or rod-in-tube method. In both cases, core and cladding glass are heated to temperatures corresponding to a viscosity range of 10⁵ to 10⁶ dPas, and are drawn out to give a fibre. In order to be able to produce a stable fibre having low attenuation, firstly, the core and cladding glass must be compatible with one another in terms of a range of properties, such as viscosity profile, thermal expansion, tendency to crystallize and more, and secondly, they must have high purity ensured, as mentioned, through pure raw materials and above all by the production method. In particular, it must be impossible for reactions between core and cladding glass, e.g. diffusion or crystallization, to occur at the interface between fibre core and fibre cladding, as these reactions disrupt the total internal reflection of the light guided in the fibre core, and thus increase attenuation. In addition, the mechanical strength of the fibre is impaired by crystallization.

Against this background, WO 2013/104748 A1 describes highly transmissive glasses that can be used as core glasses in light guides. This document focuses particularly on low attenuation in the near IR range. In particular, low attenuation at a wavelength of 1050 nm is achieved.

However, high transmission (low attenuation) at lower wavelengths is becoming increasingly important for various applications. For example, in a process referred to as radiation curing, UV-A radiation, inter alia, is used to cure materials such as paints, printing inks or adhesives. In addition to surface curing, there is also what is referred to as spot curing, in which the curing radiation is guided accurately to the desired site of action without other parts of the products to be produced being exposed to the radiation. UV spot curing is used for example in the production of cardiac catheters or oxygenators, in order to establish adhesive bonding between different materials. High transmission at low wavelengths is also becoming increasingly important for example in disinfection using UV radiation.

In this regard, there is a need for corresponding glasses, in particular for light and/or image guiding applications.

Light guide elements are also abbreviated herein to light guides. They are also often referred to as light guide fibres and given the corresponding acronym “LGF”, and image guides can accordingly be shortened to “IG”. Specific image guides are also given the acronym “LFB” for “leached fibre bundle”.

Compared to light guides, image guides comprise light guide fibres which are arranged in an ordered manner such that an input-end image can be transmitted to the output end of an image guide substantially unimpaired. In this context, reference is also commonly made to a 1:1 ordering or arrangement of light guide fibres of an image guide or in an image guide.

In a first aspect, the invention relates to a glass comprising SiO₂ and at least one of the two components Gd₂O₃ and Y₂O₃, wherein the ratio of the sum of the proportions by weight of Gd₂O₃ and Y₂O₃ to the proportion by weight of SiO₂ is at least 0.01, wherein the proportion of Ta₂O₅ is at most 10 wt %, wherein the proportion of ZrO₂ is at least 0.1 wt %, and wherein the ratio of the proportion by weight of B₂O₃ to the proportion by weight of SiO₂ is at most 0.50.

Advantageously, for a sample thickness of 25 mm and a wavelength of 380 nm, this glass has a pure transmittance of at least 0.900.

In a second aspect, the invention relates to a glass article or in particular a light guide element, in particular a glass fibre, comprising or consisting of a glass (in particular a glass of the invention) comprising SiO₂ and at least one of the two components Gd₂O₃ and Y₂O₃, wherein the ratio of the sum of the proportions by weight of Gd₂O₃ and Y₂O₃ to the proportion by weight of SiO₂ is at least 0.01, wherein the proportion of Ta₂O₅ is at most 10 wt %, wherein the proportion of ZrO₂ is at least 0.1 wt %, and wherein the ratio of the proportion by weight of B₂O₃ to the proportion by weight of SiO₂ is at most 0.50.

In a third aspect, the invention relates to a method for producing a glass (in particular a glass of the invention) comprising SiO₂ and at least one of the two components Gd₂O₃ and Y₂O₃, wherein the ratio of the sum of the proportions by weight of Gd₂O₃ and Y₂O₃ to the proportion by weight of SiO₂ is at least 0.01, wherein the proportion of Ta₂O₅ is at most 10 wt %, wherein the proportion of ZrO₂ is at least 0.1 wt %, and wherein the ratio of the proportion by weight of B₂O₃ to the proportion by weight of SiO₂ is at most 0.50, the method comprising the following steps:
melting the glass raw materials,
cooling the glass obtained, a glass of the invention being obtained in particular.

In a fourth aspect, the invention relates to a method for producing a glass article (in particular a light guide element) comprising SiO₂ and at least one of the two components Gd₂O₃ and Y₂O₃, wherein the ratio of the sum of the proportions by weight of Gd₂O₃ and Y₂O₃ to the proportion by weight of SiO₂ is at least 0.01, wherein the proportion of Ta₂O₅ is at most 10 wt %, wherein the proportion of ZrO₂ is at least 0.1 wt %, and wherein the ratio of the proportion by weight of B₂O₃ to the proportion by weight of SiO₂ is at most 0.50, the method comprising the following steps:
melting the glass raw materials,
cooling the glass obtained, a glass article, in particular a light guide element of the invention being obtained in particular.

In a fifth aspect, the invention relates to the use of a glass (in particular a glass of the invention) as fibre glass, in particular as core glass in a light and/or image guide, wherein the glass comprises SiO₂ and at least one of the two components Gd₂O₃ and Y₂O₃, wherein the ratio of the sum of the proportions by weight of Gd₂O₃ and Y₂O₃ to the proportion by weight of SiO₂ is at least 0.01, wherein the proportion of Ta₂O₅ is at most 10 wt %, wherein the proportion of ZrO₂ is at least 0.1 wt %, and wherein the ratio of the proportion by weight of B₂O₃ to the proportion by weight of SiO₂ is at most 0.50.

In a six aspect, the invention relates to the use of a glass article (in particular a glass of the invention) as fibre glass, in particular as core glass in a light and/or image guide, wherein the glass article comprises SiO₂ and at least one of the two components Gd₂O₃ and Y₂O₃, wherein the ratio of the sum of the proportions by weight of Gd₂O₃ and Y₂O₃ to the proportion by weight of SiO₂ is at least 0.01, wherein the proportion of Ta₂O₅ is at most 10 wt %, wherein the proportion of ZrO₂ is at least 0.1 wt %, and wherein the ratio of the proportion by weight of B₂O₃ to the proportion by weight of SiO₂ is at most 0.50.

In a seventh aspect, the invention relates to the use of a light guide element, in particular a light guide element in accordance with the invention, in endoscopic applications, in particular endoscopes, advantageously single-use endoscopes, in projection apparatuses, in optical data transmission technology, automotive applications, laser technology and disinfection, wherein the light guide element comprises a glass, in particular as core glass, which comprises SiO₂ and at least one of the two components Gd₂O₃ and Y₂O₃, wherein the ratio of the sum of the proportions by weight of Gd₂O₃ and Y₂O₃ to the proportion by weight of SiO₂ is at least 0.01, wherein the proportion of Ta₂O₅ is at most 10 wt %, wherein the proportion of ZrO₂ is at least 0.1 wt %, and wherein the ratio of the proportion by weight of B₂O₃ to the proportion by weight of SiO₂ is at most 0.50.

A glass in which the proportion of Y₂O₃ and Gd₂O₃ is in each case at least 0.1 wt % is advantageous, a glass in which these proportions are in each case at least 0.2 wt % is particularly advantageous. This can improve meltability. Further advantages, for example the suppression of crystallites, are also mentioned in this description.

The aspects of the invention can in particular be additionally configured by means of the embodiments described herein. The embodiments relate to all aspects of the invention and can in particular also be combined with one another.

The invention relates to redrawable glass, in particular for glass fibres. The invention also relates to methods for producing same, and to uses thereof. The glasses of the invention are preferably used as core glass in a light and/or image guide. The invention also relates to a light guide element, in particular a light and/or image guide, that comprises the glass according to the invention as core glass, and a cladding glass.

### Glass Composition

The composition of the glasses according to the invention is given here in percentage by weight (wt %), unless otherwise indicated. Unless otherwise indicated, the figures relate to the analytical compositions. Those skilled in the art are aware of how to analyse the composition of a glass. The analysis can in particular take place by means of X-ray fluorescence spectroscopy (XRF). In the present disclosure, where reference is made to the synthesis compositions, this is explicitly indicated.

In some embodiments, the proportion of SiO₂ is in a range from 10 to 55 wt %, for example in a range from 15 to 45 wt %, from 17 to 40 wt %, from 18 to 35 wt %, from 20 to 35 wt %, from 22 to 35 wt %, from 23 to 34 wt %, from 24 to 33 wt %, from 24 to 32 wt %, from 25 to 32 wt %, or from 27 to 31 wt %. In some embodiments, the proportion of SiO₂ is at least 10 wt %, for example at least 15 wt %, at least 17 wt %, at least 18 wt %, at least 20 wt %, at least 22 wt %, at least 23 wt %, at least 24 wt %, at least 25 wt %, or at least 27 wt %. In some embodiments, the proportion of SiO₂ is at most 55 wt %, at most 45 wt %, at most 40 wt %, at most 35 wt %, at most 34 wt %, at most 33 wt %, at most 32 wt %, or at most 31 wt %.

In some embodiments, the proportion of Gd₂O₃ is in a range from 0 to 15 wt %, for example from 0 to 10 wt %, from 0 to 9.0 wt %, from 0 to 8.0 wt %, from 0.1 to 7.0 wt %, from 0.2 to 7.0 wt %, from 0.5 to 7.0 wt %, from 1.0 to 7.0 wt %, from 1.5 to 6.0 wt %, from 2.0 to 5.5 wt %, from 2.5 to 5.0 wt %, from 3.0 to 5.5 wt %, from 3.0 to 4.5 wt %, or from 3.5 to 4.0 wt %. In some embodiments, the proportion of Gd₂O₃ is at least 0.1 wt %, at least 0.2 wt %, at least 0.5 wt %, at least 1.0 wt %, at least 1.5 wt %, at least 2.0 wt %, at least 2.5 wt %, at least 3.0 wt %, or at least 3.5 wt %. In some embodiments, the proportion of Gd₂O₃ is at most 15 wt %, for example at most 10 wt %, at most 9.0 wt %, at most 8.0 wt %, at most 7.0 wt %, at most 6.0 wt %, at most 5.5 wt %, at most 5.0 wt %, at most 4.5 wt %, or at most 4.0 wt %. In some embodiments, the proportion of Gd₂O₃ is at most 3.0 wt %, at most 2.0 wt %, at most 1.0 wt %, at most 0.5 wt %, at most 0.2 wt %, at most 0.1 wt %, at most 0.05 wt %, or at most 0.01 wt %. In some embodiments, the glass is free from Gd₂O₃.

In some embodiments, the proportion of Y₂O₃ is in a range from 0 to 15 wt %, for example from 0 to 10 wt %, from 1.0 to 10 wt %, from 0 to 9.0 wt %, from 0 to 8.0 wt %, from 0.1 to 7.0 wt %, from 0.2 to 5.0 wt %, from 0.5 to 2.5 wt %, from 0.5 to 2.0 wt %, from 0.5 to 1.5 wt %, from 2.0 to 8.0 wt %, or from 2.5 to 6.0 wt %. In some embodiments, the proportion of Y₂O₃ is at least 0.1 wt %, at least 0.2 wt %, at least 0.5 wt %, at least 1.0 wt %, at least 1.5 wt %, at least 2.0 wt %, or at least 2.5 wt %. In some embodiments, the proportion of Y₂O₃ is at most 15 wt %, for example at most 10 wt %, at most 9.0 wt %, at most 8.0 wt %, at most 7.0 wt %, at most 6.0 wt %, at most 5.0 wt %, at most 2.5 wt %, at most 2.0 wt %, at most 1.5 wt %, or at most 1.0 wt %, at most 0.5 wt %, at most 0.2 wt %, at most 0.1 wt %, at most 0.05 wt %, or at most 0.01 wt %. In some embodiments, the glass is free from Y₂O₃.

In some embodiments, the sum of the proportions by weight of Gd₂O₃ and Y₂O₃ is in a range from 0.2 to 20 wt %, for example from 0.5 to 15 wt %, from 1.0 to 10 wt %, from 1.5 to 9.0 wt %, from 2.0 to 8.0 wt %, from 2.5 to 7.0 wt %, from 3.0 to 6.0 wt %, from 3.5 to 5.5 wt %, or from 4.0 to 5.0 wt %. In some embodiments, the sum of the proportions by weight of Gd₂O₃ and Y₂O₃ is at least 0.2 wt %, for example at least 0.5 wt %, at least 1.0 wt %, at least 1.5 wt %, at least 2.0 wt %, at least 2.5 wt %, at least 3.0 wt %, at least 3.5 wt %, or at least 4.0 wt %. In some embodiments, the sum of the proportions by weight of Gd₂O₃ and Y₂O₃ is at most 20 wt %, for example at most 15 wt %, at most 10 wt %, at most 9.0 wt %, at most 8.0 wt %, at most 7.0 wt %, at most 6.0 wt %, at most 5.5 wt %, or at most 5.0 wt %.

In some embodiments, the ratio of the sum of the proportions by weight of Gd₂O₃ and Y₂O₃ to the proportion by weight of SiO₂ is in a range from 0.01 to 0.75, for example in a range from 0.02 to 0.60, from 0.04 to 0.50, from 0.06 to 0.40, from 0.08 to 0.30, from 0.09 to 0.25, from 0.10 to 0.20, or from 0.12 to 0.18. In some embodiments, the ratio of the sum of the proportions by weight of Gd₂O₃ and Y₂O₃ to the proportion by weight of SiO₂ is at least 0.01, for example at least 0.02, at least 0.04, at least 0.06, at least 0.08, at least 0.09, at least 0.10, or at least 0.12. In some embodiments, the ratio of the sum of the proportions by weight of Gd₂O₃ and Y₂O₃ to the proportion by weight of SiO₂ is at most 0.75, for example at most 0.60, at most 0.50, at most 0.40, at most 0.30, at most 0.25, or at most 0.20.

In some embodiments, the proportion of Ta₂O₅ is in a range from 0 to 10 wt %, for example from 0 to 9.0 wt %, from 0 to 8.0 wt %, from 0 to 7.0 wt %, from 0.1 to 7.0 wt %, from 0.2 to 7.0 wt %, from 0.5 to 7.0 wt %, from 1.0 to 7.0 wt %, from 2.0 to 7.0 wt %, from 2.0 to 5.0 wt %, from 2.5 to 6.5 wt %, from 3.0 to 6.0 wt %, from 3.5 to 5.5 wt %, from 4.0 to 5.0 wt %, from 4.1 to 4.9 wt %, or from 4.2 to 4.8 wt %. In some embodiments, the proportion of Ta₂O₅ is at least 0.1 wt %, for example at least 0.2 wt %, at least 0.5 wt %, at least 1.0 wt %, at least 2.0 wt %, at least 2.5 wt %, at least 3.0 wt %, at least 3.5 wt %, at least 4.0 wt %, at least 4.1 wt %, or at least 4.2 wt %. In some embodiments, the proportion of Ta₂O₅ is at most 10 wt %, for example at most 9.0 wt %, at most 8.0 wt %, at most 7.0 wt %, at most 6.5 wt %, at most 6.0 wt %, at most 5.5 wt %, at most 5.0 wt %, at most 4.9 wt %, at most 4.8 wt %, at most 4.5 wt %, at most 4.0 wt %, at most 3.0 wt %, at most 2.0 wt %, at most 1.0 wt %, at most 0.5 wt %, at most 0.2 wt %, at most 0.1 wt %, at most 0.05 wt %, or at most 0.01 wt %. In some particularly advantageous embodiments, the glass is free from Ta₂O₅.

In some embodiments, the sum of the proportions by weight of Ta₂O₅ and SiO₂ is in a range from 20 to 50 wt %, for example in a range from >20 to 49 wt %, from 21 to 48 wt %, from 22 to 45 wt %, from 25 to 40 wt %, from 27 to 38 wt %, from 29 to 37 wt %, from 30 to 36 wt %, or from 31 to 35 wt %. In some embodiments, the sum of the proportions by weight of Ta₂O₅ and SiO₂ is at least 20 wt %, for example more than 20 wt %, at least 21 wt %, at least 22 wt %, at least 25 wt %, at least 27 wt %, at least 29 wt %, at least 30 wt %, or at least 31 wt %. In some embodiments, the sum of the proportions by weight of Ta₂O₅ and SiO₂ is at most 50 wt %, for example at most 49 wt %, at most 48 wt %, at most 45 wt %, at most 40 wt %, at most 38 wt %, at most 37 wt %, at most 36 wt %, or at most 35 wt %.

In some embodiments, the proportion of Y₂O₃ and Gd₂O₃ is in each case at least 0.1 wt %, advantageously at least 0.2 wt % or at least 0.5 wt %. Advantageous upper limits for the sum of Y₂O₃ and Gd₂O₃ were mentioned above. In some embodiments, the ratio of the proportion by weight of Ta₂O₅ to the sum of the proportions by weight of Gd₂O₃ and Y₂O₃ is in a range from 0 to 8.0, for example from 0.10 to 6.0, from 0.20 to 5.0, in a range from 0.25 to 4.0, from 0.30 to 3.5, from 0.35 to 3.0, from 0.40 to 2.9, from 0.45 to 2.8, from 0.45 to 2.5, from 0.50 to 2.0, from 0.60 to 1.7, from 0.70 to 1.5, from 0.75 to 1.2, from 0.80 to 1.1, or from 0.90 to 1.0. In some embodiments, the ratio of the proportion by weight of Ta₂O₅ to the sum of the proportions by weight of Gd₂O₃ and Y₂O₃ is at least 0.10, for example at least 0.20, at least 0.25, at least 0.30, at least 0.35, at least 0.40, at least 0.45, at least 0.50, at least 0.60, at least 0.70, at least 0.75, at least 0.80, or at least 0.90. In some embodiments, the ratio of the proportion by weight of Ta₂O₅ to the sum of the proportions by weight of Gd₂O₃ and Y₂O₃ is at most 8.0, for example at most 6.0, at most 5.0, at most 4.0, at most 3.5, at most 3.0, at most 2.9, at most 2.8, at most 2.5, at most 2.0, at most 1.7, at most 1.5, at most 1.2, at most 1.1, at most 1.0, at most 0.8, at most 0.5, at most 0.2, at most 0.1 or even 0.

In some embodiments, the proportion of BaO is in a range from 0 to 50 wt %, for example from 0.1 to 45 wt %, from 1.0 to 35 wt %, from 2.0 to 30 wt %, from 5.0 to 30 wt %, from 10 to 30 wt %, from 15 to 30 wt %, from 15 to 28 wt %, from 17 to 27 wt %, from 17 to 26 wt %, from 17 to 21 wt %, or from 19 to 24 wt %. In some embodiments, the proportion of BaO is at least 0.1 wt %, for example at least 1.0 wt %, at least 2.0 wt %, at least 5.0 wt %, at least 10 wt %, at least 15 wt %, at least 17 wt %, or at least 19 wt %. In some embodiments, the proportion of BaO is at most 50 wt %, for example at most 45 wt %, at most 35 wt %, at most 30 wt %, at most 28 wt %, at most 27 wt %, at most 26 wt %, at most 24 wt %, or at most 21 wt %.

In some embodiments, the proportion of La₂O₃ is in a range from 0 to 70 wt %, for example from 0.1 to 50 wt %, from 2.0 to 45 wt %, from 5.0 to 40 wt %, from 10 to 30 wt %, from 15 to 25 wt %, from 15 to 24 wt %, from 17 to 22 wt %, from 17 to 28 wt %, or from 19 to 26 wt %. In some embodiments, the proportion of La₂O₃ is at least 0.1 wt %, for example at least 2.0 wt %, at least 5.0 wt %, at least 10 wt %, at least 12 wt %, at least 15 wt %, at least 16 wt %, at least 17 wt %, or at least 19 wt %. In some embodiments, the proportion of La₂O₃ is at most 70 wt %, for example at most 50 wt %, at most 45 wt %, at most 40 wt %, at most 38 wt %, at most 37 wt %, at most 35 wt %, at most 30 wt %, at most 28 wt %, at most 26 wt %, at most 25 wt %, at most 24 wt %, or at most 22 wt %.

In some embodiments, the sum of the proportions of BaO and La₂O₃ is in a range from 20 wt % to 60 wt %, for example from 25 to 55 wt %, from 30 to 50 wt %, from 32 to 48 wt %, from 35 to 45 wt %, or from 38 to 44 wt %. In some embodiments, the sum of the proportions of BaO and La₂O₃ is at least 20 wt %, for example at least 25 wt %, at least 30 wt %, at least 32 wt %, at least 35 wt %, or at least 38 wt %. In some embodiments, the sum of the proportions of BaO and La₂O₃ is at most 60 wt %, for example at most 55 wt %, at most 50 wt %, at most 48 wt %, at most 45 wt %, or at most 44 wt %.

In some embodiments, the ratio of the proportion by weight of Gd₂O₃ to the proportion by weight of La₂O₃ is in a range from 0.01 to 0.75, for example from 0.02 to 0.50, from 0.05 to 0.35, from 0.06 to 0.34, from 0.07 to 0.33, from 0.10 to 0.30, or from 0.15 to 0.25. In some embodiments, the ratio of the proportion by weight of Gd₂O₃ to the proportion by weight of La₂O₃ is at least 0.01, for example at least 0.02, at least 0.05, at least 0.06, at least 0.07, at least 0.10, or at least 0.15. In some embodiments, the ratio of the proportion by weight of Gd₂O₃ to the proportion by weight of La₂O₃ is at most 0.75, for example at most 0.50, at most 0.35, at most 0.34, at most 0.33, at most 0.30, or at most 0.25.

This means that the ratio of the proportion by weight of La₂O₃ to the sum of the proportions by weight of Gd₂O₃ and Y₂O₃ is advantageously at most 10. La₂O₃ contributes to setting a high refractive index in the glass. As a result, generally relatively high contents of this component are sought. However, it has been shown that, particularly with high proportions of La₂O₃, in particular in the range of a minimum proportion of 15 wt % or more, the meltability of the glass becomes less rational. In particular, the melting point increases and the glass tends to devitrify. The inventors have found that the presence of Gd₂O₃ and/or Y₂O₃, in particular at the minimum contents mentioned herein, can counteract devitrification. In particular, if the ratio of La₂O₃ to the sum of the proportions by weight of Gd₂O₃ and Y₂O₃ is set as described, the crystallization tendency of the glass can be suppressed. It is assumed that La₂O₃ and B₂O₃ can react to give lanthanum borates, which crystallize out. However, in particular Gd₂O₃, but also Y₂O₃, advantageously the combination of the two, stabilize the glass network, thereby reducing the crystallization tendency and improving the meltability of the glass.

The presence in particular of Y₂O₃, but also Gd₂O₃, preferably the combination of the two, can also contribute to improving absorption in the blue region of the visible spectrum, i.e. at a wavelength of approximately 400 nm, such that better transmission and less absorption occurs there.

In some embodiments, the sum of the proportions of La₂O₃ and Gd₂O₃ is in a range from 10 to 50 wt %, for example from 12 to 40 wt %, from 15 to 30 wt %, from 17 to 29 wt %, from 19 to 28 wt %, from 20 to 27 wt %, or from 21 to 26 wt %. In some embodiments, the sum of the proportions of La₂O₃ and Gd₂O₃ is at least 10 wt %, for example at least 12 wt %, at least 15 wt %, at least 17 wt %, at least 19 wt %, at least 20 wt %, or at least 21 wt %. In some embodiments, the sum of the proportions of La₂O₃ and Gd₂O₃ is at most 50 wt %, for example at most 40 wt %, at most 30 wt %, at most 29 wt %, at most 28 wt %, at most 27 wt %, or at most 26 wt %.

In some embodiments, the ratio of the proportion by weight of Y₂O₃ to the proportion by weight of La₂O₃ is in a range from 0.01 to 0.15, for example from 0.02 to 0.10, from 0.03 to 0.08, or from 0.04 to 0.06. In some embodiments, the ratio of the proportion by weight of Y₂O₃ to the proportion by weight of La₂O₃ is at least 0.01, for example at least 0.02, at least 0.03, or at least 0.04. In some embodiments, the ratio of the proportion by weight of Y₂O₃ to the proportion by weight of La₂O₃ is at most 0.15, for example at most 0.10, at most 0.08, or at most 0.06.

In some embodiments, the sum of the proportions of La₂O₃ and Y₂O₃ is in a range from 10 to 31 wt %, for example from 12 to 29 wt %, from 14 to 27 wt %, from 16 to 25 wt %, or from 18 to 23 wt %. In some embodiments, the sum of the proportions of La₂O₃ and Y₂O₃ is at least 10 wt %, for example at least 12 wt %, at least 14 wt %, at least 16 wt %, or at least 18 wt %. In some embodiments, the sum of the proportions of La₂O₃ and Y₂O₃ is at most 31 wt %, for example at most 29 wt %, at most 27 wt %, at most 25 wt %, or at most 23 wt %.

In some embodiments, the ratio of the proportion by weight of La₂O₃ to the sum of the proportions by weight of Gd₂O₃ and Y₂O₃ is in a range from 1.0 to 10, for example from 2.0 to 8.0, from 2.5 to 7.0, from 3.0 to 6.0 or from 3.5 to 5.0. In some embodiments, the ratio of the proportion by weight of La₂O₃ to the sum of the proportions by weight of Gd₂O₃ and Y₂O₃ is at least 1.0, for example at least 2.0, at least 2.5, at least 3.0, or at least 3.5. In some embodiments, the ratio of the proportion by weight of La₂O₃ to the sum of the proportions by weight of Gd₂O₃ and Y₂O₃ is at most 10, for example at most 8.0, at most 7.0, at most 6.0, or at most 5.0.

In some embodiments, the sum of the proportions of La₂O₃, Gd₂O₃ and Y₂O₃ is in a range from 10 to 50 wt %, for example from 12 to 40 wt %, from 15 to 35 wt %, from 18 to 32 wt %, from 20 to 30 wt %, from 21 to 27 wt %, or from 22 to 26 wt %. In some embodiments, the sum of the proportions of La₂O₃, Gd₂O₃ and Y₂O₃ is at least 10 wt %, for example at least 12 wt %, at least 15 wt %, at least 18 wt %, at least 20 wt %, at least 21 wt %, or at least 22 wt %. In some embodiments, the sum of the proportions of La₂O₃, Gd₂O₃ and Y₂O₃ is at most 50 wt %, for example at most 40 wt %, at most 35 wt %, at most 32 wt %, at most 30 wt %, at most 27 wt %, or at most 26 wt %.

In some embodiments, the proportion of B₂O₃ is in a range from 0 to 25 wt %, for example from 0.1 to 20 wt %, from 0.5 to 15 wt %, from 1.0 to 13 wt %, from 2.0 to 10 wt %, from 2.0 to 8.0 wt %, from 2.0 to 6.0 wt %, from 2.0 to 5.0 wt %, from 2.5 to 4.5 wt %, from 2.5 to 4.0 wt %, or from 3.0 to 4.0 wt %. In some embodiments, the proportion of B₂O₃ is at least 0.1 wt %, for example at least 0.2 wt %, at least 0.5 wt %, at least 1.0 wt %, at least 1.5 wt %, at least 2.0 wt %, at least 2.5 wt %, or at least 3.0 wt %. In some embodiments, the proportion of B₂O₃ is at most 25 wt %, for example at most 20 wt %, at most 15 wt %, at most 13 wt %, at most 10 wt %, at most 8.0 wt %, at most 6.0 wt %, at most 5.0 wt %, at most 4.5 wt %, or at most 4.0 wt %.

According to the invention, the ratio of the proportion by weight of B₂O₃ to the proportion by weight of SiO₂ is at most 0.50. In some embodiments, the ratio of the proportion by weight of B₂O₃ to the proportion by weight of SiO₂ is in a range from 0 to 0.50, for example from 0 to <0.50, from 0.01 to 0.40, from 0.02 to 0.30, from 0.04 to 0.20, from 0.05 to 0.18, from 0.06 to 0.17, from 0.08 to 0.16, from 0.10 to 0.15, or from 0.11 to 0.14. In some embodiments, the ratio of the proportion by weight of B₂O₃ to the proportion by weight of SiO₂ is at least 0.01, for example at least 0.02, at least 0.04, at least 0.05, at least 0.06, at least 0.08, at least 0.10, or at least 0.11. In some embodiments, the proportion by weight of B₂O₃ is smaller than the proportion by weight of SiO₂. In some embodiments, the ratio of the proportion by weight of B₂O₃ to the proportion by weight of SiO₂ is at most 0.50, at most 0.40, at most 0.30, at most 0.20, at most 0.18, at most 0.17, at most 0.16, at most 0.15, or at most 0.14.

In some embodiments, the sum of the proportions of SiO₂ and B₂O₃ is in a range from 15 to 50 wt %, for example from 20 to 44 wt %, from 25 to 40 wt %, from 27 to 37 wt %, from 29 to 35 wt %, or from 30 to 34 wt %. In some embodiments, the sum of the proportions of SiO₂ and B₂O₃ is at least 15 wt %, for example at least 20 wt %, at least 25 wt %, at least 27 wt %, at least 29 wt %, or at least 30 wt %. In some embodiments, the sum of the proportions of SiO₂ and B₂O₃ is at most 50 wt %, for example at most 44 wt %, at most 40 wt %, at most 37 wt %, at most 35 wt %, or at most 34 wt %.

In some embodiments, the proportion of Nb₂O₅ is in a range from 0 to 10 wt %, for example from 0 to 9.0 wt %, from 0 to 8.0 wt %, from 0 to 7.0 wt %, from 0.1 to 5.0 wt %, from 0.1 to 2.0 wt %, from 0.1 to 1.5 wt %, from 0.1 to 1.0 wt %, or from 0.1 to 0.5 wt %. In some embodiments, the proportion of Nb₂O₅ is at least 0.1 wt %, for example at least 0.2 wt % or at least 0.5 wt %. In some embodiments, the proportion of Nb₂O₅ is at most 10 wt %, for example at most 9.0 wt %, at most 8.0 wt %, at most 7.0 wt %, at most 5.0 wt %, at most 2.0 wt %, at most 1.5 wt %, at most 1.0 wt %, at most 0.5 wt %, at most 0.2 wt %, at most 0.1 wt %, at most 0.05 wt %, or at most 0.01 wt %. In some embodiments, the glass is free from Nb₂O₅.

In some embodiments, the sum of the proportions of La₂O₃, Gd₂O₃, Ta₂O₅ and Nb₂O₅ is in a range from 15 to 50 wt %, for example from 20 to 45 wt %, from >20 to 40 wt %, from 21 to 38 wt %, from 22 to 36 wt %, from 23 to 34 wt %, from 24 to 32 wt %, or from 25 to 31 wt %. In some embodiments, the sum of the proportions of La₂O₃, Gd₂O₃, Ta₂O₅ and Nb₂O₅ is at least 15 wt %, for example at least 20 wt %, more than 20 wt %, at least 21 wt %, at least 22 wt %, at least 23 wt %, at least 24 wt %, or at least 25 wt %. In some embodiments, the sum of the proportions of La₂O₃, Gd₂O₃, Ta₂O₅ and Nb₂O₅ is at most 50 wt %, at most 45 wt %, at most 40 wt %, at most 38 wt %, at most 36 wt %, at most 34 wt %, at most 32 wt %, or at most 31 wt %.

In some embodiments, the proportion by weight of Y₂O₃ is greater than the proportion of Nb₂O₅. In some embodiments, the ratio of the proportion by weight of Nb₂O₅ to the proportion by weight of Y₂O₃ is in a range from 0 to <1.00, for example from 0.01 to 0.90, from 0.02 to 0.75, from 0.05 to 0.50, from 0.10 to 0.35, or from 0.15 to 0.25. In some embodiments, the ratio of the proportion by weight of Nb₂O₅ to the proportion by weight of Y₂O₃ is at least 0.01, for example at least 0.02, at least 0.05, at least 0.10, or at least 0.15. In some embodiments, the ratio of the proportion by weight of Nb₂O₅ to the proportion by weight of Y₂O₃ is less than 1.00. In some embodiments, the ratio of the proportion by weight of Nb₂O₅ to the proportion by weight of Y₂O₃ is at most 0.90, at most 0.75, at most 0.50, at most 0.35, or at most 0.25, for example at most 0.15, at most 0.10, at most 0.05, at most 0.02, at most 0.01, or even 0.

In some embodiments, the sum of the proportions of Ta₂O₅ and Nb₂O₅ is in a range from 0 to 10 wt %, for example from 0.1 to 9.5 wt %, from 0.2 to 9.0 wt %, from 0.5 to 8.5 wt %, from 1.0 to 8.0 wt %, from 1.5 to 7.5 wt %, from 2.0 to 7.0 wt %, from 2.5 to 7.0 wt %, from 3.0 to 7.0 wt %, from 3.0 to 6.5 wt %, from 3.5 to 5.5 wt %, from 4.0 to 5.0 wt %, from 4.1 to 4.9 wt %, or from 4.2 to 4.8 wt %. In some embodiments, the sum of the proportions of Ta₂O₅ and Nb₂O₅ is at least 0.1 wt %, for example at least 0.2 wt %, at least 0.5 wt %, at least 1.0 wt %, at least 2.0 wt %, at least 2.5 wt %, at least 3.0 wt %, at least 3.5 wt %, at least 4.0 wt %, at least 4.1 wt %, or at least 4.2 wt %. In some embodiments, the sum of the proportions of Ta₂O₅ and Nb₂O₅ is at most 10 wt %, for example at most 9.5 wt %, at most 9.0 wt %, at most 8.5 wt %, at most 8.0 wt %, at most 7.5 wt %, at most 7.0 wt %, at most 6.5 wt %, at most 6.0 wt %, at most 5.5 wt %, at most 5.0 wt %, at most 4.9 wt %, at most 4.8 wt %, at most 4.5 wt %, at most 4.0 wt %, at most 3.0 wt %, at most 2.5 wt %, at most 2.0 wt %, at most 1.5 wt %, or at most 1.0 wt %.

In some embodiments, the ratio of the proportion by weight of Nb₂O₅ to the proportion by weight of Ta₂O₅ is in a range from 0 to 0.30, for example from 0 to 0.25, from 0.01 to 0.20, from 0.02 to 0.10, or from 0.03 to 0.05. In some embodiments, the ratio of the proportion by weight of Nb₂O₅ to the proportion by weight of Ta₂O₅ is at least 0.01, at least 0.02, or at least 0.03. In some embodiments, the ratio of the proportion by weight of Nb₂O₅ to the proportion by weight of Ta₂O₅ is at most 0.30, for example at most 0.25, at most 0.20, at most 0.15, at most 0.10, at most 0.08, at most 0.05, at most 0.02, at most 0.01, or even 0.

In some embodiments, the ratio of the proportion by weight of Nb₂O₅ to the sum of the proportions by weight of Gd₂O₃ and Y₂O₃ is in a range from 0 to 0.30, for example from 0 to 0.25, from 0.01 to 0.20, from 0.02 to 0.10 or from 0.03 to 0.05. In some embodiments, the ratio of the proportion by weight of Nb₂O₅ to the sum of the proportions by weight of Gd₂O₃ and Y₂O₃ is at least 0.01, at least 0.02, or at least 0.03. In some embodiments, the ratio of the proportion by weight of Nb₂O₅ to the sum of the proportions by weight of Gd₂O₃ and Y₂O₃ is at most 0.30, for example at most 0.25, at most 0.20, at most 0.15, at most 0.10, at most 0.08, at most 0.05, at most 0.02, at most 0.01, or even 0.

According to the invention, the glass contains ZrO₂. The lower limit is 0.1 wt %. In some embodiments, the proportion of ZrO₂ is in a range from 0.1 to 10 wt %, for example from 0.1 to 9.0 wt %, from 0.5 to 8.0 wt %, from 1.0 to 7.0 wt %, from 1.0 to 5.0 wt %, from 1.5 to 4.5 wt %, from 2.0 to 4.5 wt %, from 2.0 to 3.5 wt %, or from 2.5 to 4.0 wt %. In some embodiments, the proportion of ZrO₂ is at least 0.1 wt %, at least 0.2 wt %, at least 0.5 wt %, at least 1.0 wt %, at least 1.5 wt %, at least 2.0 wt %, or at least 2.5 wt %. In some embodiments, the proportion of ZrO₂ is at most 10 wt %, for example at most 9.0 wt %, at most 8.0 wt %, at most 7.0 wt %, at most 5.0 wt %, at most 4.5 wt %, at most 4.0 wt %, or at most 3.5 wt %.

In some embodiments, the sum of the proportions by weight of Nb₂O₅ and ZrO₂ is in a range from 0 to 15 wt %, for example from 0.1 to 12 wt %, from 0.2 to 10 wt %, from 0.5 to 8.0 wt %, from 1.0 to 7.0 wt %, from 1.5 to 6.0 wt %, from 2.0 to 5.0 wt %, or from 2.5 to 4.0 wt %. In some embodiments, the sum of the proportions by weight of Nb₂O₅ and ZrO₂ is at least 0.1 wt %, for example at least 0.2 wt %, at least 0.5 wt %, at least 1.0 wt %, at least 1.5 wt %, at least 2.0 wt %, or at least 2.5 wt %. In some embodiments, the sum of the proportions by weight of Nb₂O₅ and ZrO₂ is at most 15 wt %, for example at most 12 wt %, at most 10 wt %, at most 8.0 wt %, at most 7.0 wt %, at most 6.0 wt %, at most 5.0 wt %, or at most 4.0 wt %.

In some embodiments, the ratio of the proportion by weight of Nb₂O₅ to the proportion by weight of ZrO₂ is in a range from 0.01 to 0.50, for example from 0.02 to 0.30, from 0.03 to 0.20, or from 0.04 to 0.10. In some embodiments, the ratio of the proportion by weight of Nb₂O₅ to the proportion by weight of ZrO₂ is at least 0.01, for example at least 0.02, at least 0.03, or at least 0.04. In some embodiments, the ratio of the proportion by weight of Nb₂O₅ to the proportion by weight of ZrO₂ is at most 0.50, for example at most 0.30, at most 0.20, at most 0.10, at most 0.07, at most 0.05, at most 0.02, at most 0.01, or even 0.

In some embodiments, the proportion of Li₂O is in a range from 0 to 5.0 wt %, for example from 0 to 2.0 wt %, from 0 to 1.5 wt %, from 0 to 1.0 wt %, from 0.1 to 1.0 wt %, from 0.2 to 0.9 wt %, from 0.3 to 0.9 wt %, from 0.4 to 0.9 wt %, from 0.5 to 0.9 wt %, from 0.5 to 1.5 wt %, from 0.5 to 1.1 wt %, from 0.6 to 0.9 wt %, or from 0.6 to 0.8 wt %. In some embodiments, the proportion of Li₂O is at least 0.1 wt %, for example at least 0.2 wt %, at least 0.3 wt %, at least 0.4 wt %, at least 0.5 wt %, or at least 0.6 wt %. In some embodiments, the proportion of Li₂O is at most 5.0 wt %, for example at most 2.0 wt %, at most 1.5 wt %, at most 1.1 wt %, at most 1.0 wt %, at most 0.9 wt %, at most 0.8 wt %, at most 0.5 wt %, at most 0.3 wt %, at most 0.2 wt %, at most 0.1 wt %, at most 0.05 wt %, or at most 0.01 wt %. In some embodiments, the glass is free from Li₂O.

In some embodiments, the proportion of Na₂O is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the proportion of Na₂O is at least 0.1 wt %, for example at least 0.2 wt %, at least 0.3 wt %, at least 0.4 wt %, at least 0.5 wt %, or at least 0.6 wt %. In some embodiments, the proportion of Na₂O is at most 5.0 wt %, for example at most 2.0 wt %, at most 1.5 wt %, at most 1.0 wt %, at most 0.5 wt %, at most 0.3 wt %, at most 0.2 wt %, at most 0.1 wt %, at most 0.05 wt %, or at most 0.01 wt %. In some embodiments, the glass is free from Na₂O.

In some embodiments, the sum of the proportions of Li₂O and Na₂O is in a range from 0 to 10 wt %, for example from 0 to 5.0 wt %, from 0 to 4.0 wt %, from 0 to 3.0 wt %, from 0 to 2.0 wt %, from 0.1 to 1.5 wt %, from 0.1 to 1.2 wt %, from 0.1 to 1.1 wt %, or from 0.2 to 1.0 wt %. In some embodiments, the sum of the proportions of Li₂O and Na₂O is at least 0.1 wt %, for example at least 0.2 wt %, at least 0.3 wt %, at least 0.4 wt %, at least 0.5 wt %, or at least 0.6 wt %. In some embodiments, the sum of the proportions of Li₂O and Na₂O is at most 10 wt %, for example at most 5.0 wt %, at most 4.0 wt %, at most 3.0 wt %, at most 2.0 wt %, at most 1.5 wt %, at most 1.2 wt %, at most 1.1 wt %, at most 1.0 wt %, at most 0.5 wt %, at most 0.3 wt %, at most 0.2 wt %, at most 0.1 wt %, at most 0.05 wt %, or at most 0.01 wt %. In some embodiments, the glass is free from Li₂O and Na₂O.

In some embodiments, the proportion of K₂O is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the proportion of K₂O is at least 0.1 wt %, for example at least 0.2 wt %, at least 0.3 wt %, at least 0.4 wt %, at least 0.5 wt %, or at least 0.6 wt %. In some embodiments, the proportion of K₂O is at most 5.0 wt %, for example at most 2.0 wt %, at most 1.5 wt %, at most 1.0 wt %, at most 0.5 wt %, at most 0.3 wt %, at most 0.2 wt %, at most 0.1 wt %, at most 0.05 wt %, or at most 0.01 wt %. In some embodiments, the glass is free from K₂O.

In some embodiments, the sum of the proportions of Na₂O and K₂O is in a range from 0 to 10 wt %, for example from 0 to 5.0 wt %, from 0 to 4.0 wt %, from 0 to 3.0 wt %, from 0 to 2.0 wt %, from 0.1 to 1.5 wt %, from 0.1 to 1.2 wt %, from 0.1 to 1.1 wt %, or from 0.2 to 1.0 wt %. In some embodiments, the sum of the proportions of Na₂O and K₂O is at least 0.1 wt %, for example at least 0.2 wt %, at least 0.3 wt %, at least 0.4 wt %, at least 0.5 wt %, or at least 0.6 wt %. In some embodiments, the sum of the proportions of Na₂O and K₂O is at most 10 wt %, for example at most 5.0 wt %, at most 4.0 wt %, at most 3.0 wt %, at most 2.0 wt %, at most 1.5 wt %, at most 1.2 wt %, at most 1.1 wt %, at most 1.0 wt %, at most 0.5 wt %, at most 0.3 wt %, at most 0.2 wt %, at most 0.1 wt %, at most 0.05 wt %, or at most 0.01 wt %. In some embodiments, the glass is free from Na₂O and K₂O.

In some embodiments, the sum of the proportions of Li₂O, Na₂O and K₂O is in a range from 0 to 10 wt %, for example from 0 to 5.0 wt %, from 0 to 4.0 wt %, from 0 to 3.0 wt %, from 0 to 2.0 wt %, from 0.1 to 1.5 wt %, from 0.1 to 1.2 wt %, from 0.1 to 1.1 wt %, or from 0.2 to 1.0 wt %. In some embodiments, the sum of the proportions of Li₂O, Na₂O and K₂O is at least 0.1 wt %, for example at least 0.2 wt %, at least 0.3 wt %, at least 0.4 wt %, at least 0.5 wt %, or at least 0.6 wt %. In some embodiments, the sum of the proportions of Li₂O, Na₂O and K₂O is at most 10 wt %, for example at most 5.0 wt %, at most 4.0 wt %, at most 3.0 wt %, at most 2.0 wt %, at most 1.5 wt %, at most 1.2 wt %, at most 1.1 wt %, at most 1.0 wt %, at most 0.5 wt %, at most 0.3 wt %, at most 0.2 wt %, at most 0.1 wt %, at most 0.05 wt %, or at most 0.01 wt %. In some embodiments, the glass is free from Li₂O, Na₂O and K₂O.

In some embodiments, the proportion of Rb₂O is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the proportion of Rb₂O is at least 0.1 wt %, for example at least 0.2 wt %, at least 0.3 wt %, at least 0.4 wt %, at least 0.5 wt %, or at least 0.6 wt %. In some embodiments, the proportion of Rb₂O is at most 5.0 wt %, for example at most 2.0 wt %, at most 1.5 wt %, at most 1.0 wt %, at most 0.5 wt %, at most 0.3 wt %, at most 0.2 wt %, at most 0.1 wt %, at most 0.05 wt %, or at most 0.01 wt %. In some embodiments, the glass is free from Rb₂O.

In some embodiments, the sum of the proportions of Li₂O, Na₂O, K₂O and Rb₂O is in a range from 0 to 10 wt %, for example from 0 to 5.0 wt %, from 0 to 4.0 wt %, from 0 to 3.0 wt %, from 0 to 2.0 wt %, from 0.1 to 1.5 wt %, from 0.1 to 1.2 wt %, from 0.1 to 1.1 wt %, or from 0.2 to 1.0 wt %. In some embodiments, the sum of the proportions of Li₂O, Na₂O, K₂O and Rb₂O is at least 0.1 wt %, for example at least 0.2 wt %, at least 0.3 wt %, at least 0.4 wt %, at least 0.5 wt %, or at least 0.6 wt %. In some embodiments, the sum of the proportions of Li₂O, Na₂O, K₂O and Rb₂O is at most 10 wt %, for example at most 5.0 wt %, at most 4.0 wt %, at most 3.0 wt %, at most 2.0 wt %, at most 1.5 wt %, at most 1.2 wt %, at most 1.1 wt %, at most 1.0 wt %, at most 0.5 wt %, at most 0.3 wt %, at most 0.2 wt %, at most 0.1 wt %, at most 0.05 wt %, or at most 0.01 wt %. In some embodiments, the glass is free from Li₂O, Na₂O, K₂O and Rb₂O.

In some embodiments, the proportion of Cs₂O is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the proportion of Cs₂O is at least 0.1 wt %, for example at least 0.2 wt %, at least 0.3 wt %, at least 0.4 wt %, at least 0.5 wt %, or at least 0.6 wt %. In some embodiments, the proportion of Cs₂O is at most 5.0 wt %, for example at most 2.0 wt %, at most 1.5 wt %, at most 1.0 wt %, at most 0.5 wt %, at most 0.3 wt %, at most 0.2 wt %, at most 0.1 wt %, at most 0.05 wt %, or at most 0.01 wt %. In some embodiments, the glass is free from Cs₂O.

In some embodiments, the sum of the proportions of Li₂O, Na₂O, K₂O and Cs₂O is in a range from 0 to 10 wt %, for example from 0 to 5.0 wt %, from 0 to 4.0 wt %, from 0 to 3.0 wt %, from 0 to 2.0 wt %, from 0.1 to 1.5 wt %, from 0.1 to 1.2 wt %, from 0.1 to 1.1 wt %, or from 0.2 to 1.0 wt %. In some embodiments, the sum of the proportions of Li₂O, Na₂O, K₂O and Cs₂O is at least 0.1 wt %, for example at least 0.2 wt %, at least 0.3 wt %, at least 0.4 wt %, at least 0.5 wt %, or at least 0.6 wt %. In some embodiments, the sum of the proportions of Li₂O, Na₂O, K₂O and Cs₂O is at most 10 wt %, for example at most 5.0 wt %, at most 4.0 wt %, at most 3.0 wt %, at most 2.0 wt %, at most 1.5 wt %, at most 1.2 wt %, at most 1.1 wt %, at most 1.0 wt %, at most 0.5 wt %, at most 0.3 wt %, at most 0.2 wt %, at most 0.1 wt %, at most 0.05 wt %, or at most 0.01 wt %. In some embodiments, the glass is free from Li₂O, Na₂O, K₂O and Cs₂O.

In some embodiments, the sum of the proportions of Na₂O, K₂O and Cs₂O is in a range from 0 to 10 wt %, for example from 0 to 5.0 wt %, from 0 to 4.0 wt %, from 0 to 3.0 wt %, from 0 to 2.0 wt %, from 0.1 to 1.5 wt %, from 0.1 to 1.2 wt %, from 0.1 to 1.1 wt %, or from 0.2 to 1.0 wt %. In some embodiments, the sum of the proportions of Na₂O, K₂O and Cs₂O is at least 0.1 wt %, for example at least 0.2 wt %, at least 0.3 wt %, at least 0.4 wt %, at least 0.5 wt %, or at least 0.6 wt %. In some embodiments, the sum of the proportions of Na₂O, K₂O and Cs₂O is at most 10 wt %, for example at most 5.0 wt %, at most 4.0 wt %, at most 3.0 wt %, at most 2.0 wt %, at most 1.5 wt %, at most 1.2 wt %, at most 1.1 wt %, at most 1.0 wt %, at most 0.5 wt %, at most 0.3 wt %, at most 0.2 wt %, at most 0.1 wt %, at most 0.05 wt %, or at most 0.01 wt %. In some embodiments, the glass is free from Na₂O, K₂O and Cs₂O.

In some embodiments, the sum of the proportions of Na₂O, K₂O, Rb₂O and Cs₂O is in a range from 0 to 10 wt %, for example from 0 to 5.0 wt %, from 0 to 4.0 wt %, from 0 to 3.0 wt %, from 0 to 2.0 wt %, from 0.1 to 1.5 wt %, from 0.1 to 1.2 wt %, from 0.1 to 1.1 wt %, or from 0.2 to 1.0 wt %. In some embodiments, the sum of the proportions of Na₂O, K₂O, Rb₂O and Cs₂O is at least 0.1 wt %, for example at least 0.2 wt %, at least 0.3 wt %, at least 0.4 wt %, at least 0.5 wt %, or at least 0.6 wt %. In some embodiments, the sum of the proportions of Na₂O, K₂O, Rb₂O and Cs₂O is at most 10 wt %, for example at most 5.0 wt %, at most 4.0 wt %, at most 3.0 wt %, at most 2.0 wt %, at most 1.5 wt %, at most 1.2 wt %, at most 1.1 wt %, at most 1.0 wt %, at most 0.5 wt %, at most 0.3 wt %, at most 0.2 wt %, at most 0.1 wt %, at most 0.05 wt %, or at most 0.01 wt %. In some embodiments, the glass is free from Na₂O, K₂O, Rb₂O and Cs₂O.

The expressions “R₂O” or “Σ R₂O” here denote the sum of the alkali metal oxides, i.e. the sum of Li₂O, Na₂O, K₂O, Rb₂O and Cs₂O. The proportion of R₂O is therefore the sum of the proportions of Li₂O, Na₂O, K₂O, Rb₂O and Cs₂O.

In some embodiments, the proportion of R₂O is in a range from 0 to 10 wt %, for example from 0 to 5.0 wt %, from 0 to 4.0 wt %, from 0 to 3.0 wt %, from 0 to 2.0 wt %, from 0.1 to 1.5 wt %, from 0.1 to 1.2 wt %, from 0.1 to 1.1 wt %, or from 0.2 to 1.0 wt %. In some embodiments, the proportion of R₂O is at least 0.1 wt %, for example at least 0.2 wt %, at least 0.3 wt %, at least 0.4 wt %, at least 0.5 wt %, or at least 0.6 wt %. In some embodiments, the proportion of R₂O is at most 10 wt %, for example at most 5.0 wt %, at most 4.0 wt %, at most 3.0 wt %, at most 2.0 wt %, at most 1.5 wt %, at most 1.2 wt %, at most 1.1 wt %, at most 1.0 wt %, at most 0.5 wt %, at most 0.3 wt %, at most 0.2 wt %, at most 0.1 wt %, at most 0.05 wt %, or at most 0.01 wt %. In some embodiments, the glass is free from R₂O.

In some embodiments, the ratio of the proportion by weight of Li₂O to the sum of the proportions by weight of Li₂O, Na₂O, K₂O, Rb₂O and Cs₂O is in a range from 0.1 to 1.0, for example from 0.3 to 0.9, from 0.5 to 0.8, or from 0.6 to 0.7. In some embodiments, the ratio of the proportion by weight of Li₂O to the sum of the proportions by weight of Li₂O, Na₂O, K₂O, Rb₂O and Cs₂O is at least 0.1, for example at least 0.1, at least 0.3, at least 0.5, at least 0.6, at least 0.7, at least 0.8, at least 0.9, or even 1.0. In some embodiments, the ratio of the proportion by weight of Li₂O to the sum of the proportions by weight of Li₂O, Na₂O, K₂O, Rb₂O and Cs₂O is at most 0.9, for example at most 0.8, at most 0.7, at most 0.6, at most 0.5, at most 0.3, at most 0.2, at most 0.1, or even 0.

In some embodiments, the ratio of the proportion by weight of Na₂O to the sum of the proportions by weight of Li₂O, Na₂O, K₂O, Rb₂O and Cs₂O is in a range from 0 to 0.7, for example from 0 to 0.5, from 0 to 0.4, or from 0.1 to 0.3. In some embodiments, the ratio of the proportion by weight of Na₂O to the sum of the proportions by weight of Li₂O, Na₂O, K₂O, Rb₂O and Cs₂O is at least 0.1, for example at least 0.2, or at least 0.3. In some embodiments, the ratio of the proportion by weight of Na₂O to the sum of the proportions by weight of Li₂O, Na₂O, K₂O, Rb₂O and Cs₂O is at most 0.7, at most 0.5, at most 0.4, at most 0.3, at most 0.2, at most 0.1, or even 0.

In some embodiments, the ratio of the proportion by weight of K₂O to the sum of the proportions by weight of Li₂O, Na₂O, K₂O, Rb₂O and Cs₂O is in a range from 0 to 0.7, for example from 0 to 0.5, from 0 to 0.4, or from 0.1 to 0.3. In some embodiments, the ratio of the proportion by weight of K₂O to the sum of the proportions by weight of Li₂O, Na₂O, K₂O, Rb₂O and Cs₂O is at least 0.1, for example at least 0.2, or at least 0.3. In some embodiments, the ratio of the proportion by weight of K₂O to the sum of the proportions by weight of Li₂O, Na₂O, K₂O, Rb₂O and Cs₂O is at most 0.7, at most 0.5, at most 0.4, at most 0.3, at most 0.2, at most 0.1, or even 0.

In some embodiments, the ratio of the proportion by weight of Rb₂O to the sum of the proportions by weight of Li₂O, Na₂O, K₂O, Rb₂O and Cs₂O is in a range from 0 to 0.7, for example from 0 to 0.5, from 0 to 0.4, or from 0.1 to 0.3. In some embodiments, the ratio of the proportion by weight of Rb₂O to the sum of the proportions by weight of Li₂O, Na₂O, K₂O, Rb₂O and Cs₂O is at least 0.1, for example at least 0.2, or at least 0.3. In some embodiments, the ratio of the proportion by weight of Rb₂O to the sum of the proportions by weight of Li₂O, Na₂O, K₂O, Rb₂O and Cs₂O is at most 0.7, at most 0.5, at most 0.4, at most 0.3, at most 0.2, at most 0.1, or even 0.

In some embodiments, the ratio of the proportion by weight of Cs₂O to the sum of the proportions by weight of Li₂O, Na₂O, K₂O, Rb₂O and Cs₂O is in a range from 0 to 0.7, for example from 0 to 0.5, from 0 to 0.4, or from 0.1 to 0.3. In some embodiments, the ratio of the proportion by weight of Cs₂O to the sum of the proportions by weight of Li₂O, Na₂O, K₂O, Rb₂O and Cs₂O is at least 0.1, for example at least 0.2, or at least 0.3. In some embodiments, the ratio of the proportion by weight of Cs₂O to the sum of the proportions by weight of Li₂O, Na₂O, K₂O, Rb₂O and Cs₂O is at most 0.7, at most 0.5, at most 0.4, at most 0.3, at most 0.2, at most 0.1, or even 0.

In some embodiments, the ratio of the sum of the proportions by weight of Na₂O and K₂O to the proportion by weight of Li₂O is in a range from 0 to 2.0, for example from 0 to 1.5, from 0 to 1.0, from 0.1 to 0.7, or from 0.2 to 0.5. In some embodiments, the ratio of the sum of the proportions by weight of Na₂O and K₂O to the proportion by weight of Li₂O is at least 0.1, for example at least 0.2, at least 0.3, or at least 0.5. In some embodiments, the ratio of the sum of the proportions by weight of Na₂O and K₂O to the proportion by weight of Li₂O is at most 2.0, for example at most 1.5, at most 1.0, at most 0.7, at most 0.5, at most 0.3, at most 0.2, at most 0.1, or even 0.

In some embodiments, the ratio of the proportion by weight of Na₂O to the proportion by weight of Li₂O is in a range from 0 to 2.0, for example from 0 to 1.5, from 0 to 1.0, from 0.1 to 0.7, or from 0.2 to 0.5. In some embodiments, the ratio of the proportion by weight of Na₂O to the proportion by weight of Li₂O is at least 0.1, for example at least 0.2, at least 0.3, or at least 0.5. In some embodiments, the ratio of the proportion by weight of Na₂O to the proportion by weight of Li₂O is at most 2.0, for example at most 1.5, at most 1.0, at most 0.7, at most 0.5, at most 0.3, at most 0.2, at most 0.1, or even 0.

In some embodiments, the ratio of the proportion by weight of K₂O to the proportion by weight of Li₂O is in a range from 0 to 2.0, for example from 0 to 1.5, from 0 to 1.0, from 0.1 to 0.7, or from 0.2 to 0.5. In some embodiments, the ratio of the proportion by weight of K₂O to the proportion by weight of Li₂O is at least 0.1, for example at least 0.2, at least 0.3, or at least 0.5. In some embodiments, the ratio of the proportion by weight of K₂O to the proportion by weight of Li₂O is at most 2.0, for example at most 1.5, at most 1.0, at most 0.7, at most 0.5, at most 0.3, at most 0.2, at most 0.1, or even 0.

In some embodiments, the proportion of MgO is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the proportion of MgO is at least 0.1 wt %, for example at least 0.2 wt %, or at least 0.3 wt %. In some embodiments, the proportion of MgO is at most 5.0 wt %, for example at most 2.0 wt %, at most 1.0 wt %, at most 0.5 wt %, at most 0.2 wt %, at most 0.1 wt %, at most 0.05 wt %, or at most 0.01 wt %. In some embodiments, the glass is free from MgO.

In some embodiments, the proportion of CaO is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the proportion of CaO is at least 0.1 wt %, for example at least 0.2 wt %, or at least 0.3 wt %. In some embodiments, the proportion of CaO is at most 5.0 wt %, for example at most 2.0 wt %, at most 1.0 wt %, at most 0.5 wt %, at most 0.2 wt %, at most 0.1 wt %, at most 0.05 wt %, or at most 0.01 wt %. In some embodiments, the glass is free from CaO.

In some embodiments, the proportion of SrO is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the proportion of SrO is at least 0.1 wt %, for example at least 0.2 wt %, or at least 0.3 wt %. In some embodiments, the proportion of SrO is at most 5.0 wt %, for example at most 2.0 wt %, at most 1.0 wt %, at most 0.5 wt %, at most 0.2 wt %, at most 0.1 wt %, at most 0.05 wt %, or at most 0.01 wt %. In some embodiments, the glass is free from SrO.

In some embodiments, the sum of the proportions of MgO, CaO and SrO is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the sum of the proportions of MgO, CaO and SrO is at least 0.1 wt %, for example at least 0.2 wt %, or at least 0.3 wt %. In some embodiments, the sum of the proportions of MgO, CaO and SrO is at most 5.0 wt %, for example at most 2.0 wt %, at most 1.0 wt %, at most 0.5 wt %, at most 0.2 wt %, at most 0.1 wt %, at most 0.05 wt %, or at most 0.01 wt %. In some embodiments, the glass is free from MgO, CaO and SrO.

In some embodiments, the ratio of the sum of the proportions by weight of CaO and SrO to the proportion by weight of BaO is in a range from 0 to 0.20, for example from 0 to 0.15, from 0 to 0.10, or from 0.01 to 0.05. In some embodiments, the ratio of the sum of the proportions by weight of CaO and SrO to the proportion by weight of BaO is at least 0.01, for example at least 0.02, or at least 0.05. In some embodiments, the ratio of the sum of the proportions by weight of CaO and SrO to the proportion by weight of BaO is at most 0.20, for example at most 0.15, at most 0.10, at most 0.05, at most 0.02, at most 0.01, or even 0.

In some embodiments, the ratio of the sum of the proportions by weight of MgO, CaO and SrO to the proportion by weight of BaO is in a range from 0 to 0.20, for example from 0 to 0.15, from 0 to 0.10, or from 0.01 to 0.05. In some embodiments, the ratio of the sum of the proportions by weight of MgO, CaO and SrO to the proportion by weight of BaO is at least 0.01, for example at least 0.02, or at least 0.05. In some embodiments, the ratio of the sum of the proportions by weight of MgO, CaO and SrO to the proportion by weight of BaO is at most 0.20, for example at most 0.15, at most 0.10, at most 0.05, at most 0.02, at most 0.01, or even 0.

In some embodiments, the sum of the proportions of MgO, CaO, SrO and BaO is in a range from 0 to 50 wt %, for example from 0.1 to 45 wt %, from 1.0 to 35 wt %, from 2.0 to 30 wt %, from 5.0 to 30 wt %, from 10 to 30 wt %, from 15 to 28 wt %, from >15 to 27 wt %, from 17 to 27 wt %, from 17 to 26 wt %, or from 19 to 24 wt %. In some embodiments, the sum of the proportions of MgO, CaO, SrO and BaO is at least 0.1 wt %, for example at least 1.0 wt %, at least 2.0 wt %, at least 5.0 wt %, at least 10 wt %, at least 15 wt %, more than 15 wt %, at least 16 wt %, at least 17 wt %, or at least 19 wt %. In some embodiments, the sum of the proportions of MgO, CaO, SrO and BaO is at most 50 wt %, for example at most 45 wt %, at most 35 wt %, at most 30 wt %, at most 28 wt %, at most 27 wt %, at most 26 wt %, or at most 24 wt %.

In some embodiments, the sum of the proportions of CaO, SrO and BaO is in a range from 0 to 50 wt %, for example from 0.1 to 45 wt %, from 1.0 to 35 wt %, from 2.0 to 30 wt %, from 5.0 to 30 wt %, from 10 to 30 wt %, from 15 to 28 wt %, from >15 to 27 wt %, from 17 to 27 wt %, from 17 to 26 wt %, or from 19 to 24 wt %. In some embodiments, the sum of the proportions of CaO, SrO and BaO is at least 0.1 wt %, for example at least 1.0 wt %, at least 2.0 wt %, at least 5.0 wt %, at least 10 wt %, at least 15 wt %, more than 15 wt %, at least 16 wt %, at least 17 wt %, or at least 19 wt %. In some embodiments, the sum of the proportions of CaO, SrO and BaO is at most 50 wt %, for example at most 45 wt %, at most 35 wt %, at most 30 wt %, at most 28 wt %, at most 27 wt %, at most 26 wt %, or at most 24 wt %.

In some embodiments, the proportion of ZnO is in a range from 0 to 30 wt %, for example from 0.1 to 25 wt %, from 0.5 to 20 wt %, from 1.0 to 18 wt %, from 2.0 to 18 wt %, from 5.0 to 18 wt %, from 5.0 to 15 wt %, from 7.0 to 14 wt %, from 10 to 18 wt %, from 11 to 17 wt %, from 11 to 16 wt %, from 12 to 15 wt %, from 12 to 15.0 wt %, from 12 to <15.0 wt %, or from 12 to 14.5 wt %. In some embodiments, the proportion of ZnO is at least 0.1 wt %, for example at least 0.5 wt %, at least 1.0 wt %, at least 2.0 wt %, at least 5.0 wt %, at least 7.0 wt %, at least 8.0 wt %, at least 10 wt %, at least 11 wt %, or at least 12 wt %. In some embodiments, the proportion of ZnO is at most 30 wt %, for example at most 25 wt %, at most 20 wt %, at most 18 wt %, at most 17 wt %, at most 16 wt %, at most 15 wt %, at most 15.0 wt %, less than 15.0 wt %, at most 14.5 wt %, or at most 14.0 wt %.

In some embodiments, the sum of the proportions of MgO, CaO, SrO, BaO and ZnO is in a range from 0 to 70 wt %, for example from 0.1 to 65 wt %, from 1.0 to 60 wt %, from 2.0 to 55 wt %, from 5.0 to 50 wt %, from 10 to 45 wt %, from 15 to 42 wt %, from 20 to 41 wt %, from 25 to 40 wt %, from 30 to 39 wt %, from 31 to 38 wt %, or from 32 to 37 wt %. In some embodiments, the sum of the proportions of MgO, CaO, SrO, BaO and ZnO is at least 0.1 wt %, for example at least 1.0 wt %, at least 2.0 wt %, at least 5.0 wt %, at least 10 wt %, at least 15 wt %, at least 20 wt %, at least 25 wt %, at least 30 wt %, at least 31 wt %, or at least 32 wt %. In some embodiments, the sum of the proportions of MgO, CaO, SrO, BaO and ZnO is at most 70 wt %, for example at most 65 wt %, at most 60 wt %, at most 55 wt %, at most 50 wt %, at most 45 wt %, at most 42 wt %, at most 41 wt %, at most 40 wt %, at most 39 wt %, at most 38 wt %, or at most 37 wt %.

In some embodiments, the sum of the proportions of BaO and ZnO is in a range from 0 to 70 wt %, for example from 0.1 to 65 wt %, from 1.0 to 60 wt %, from 2.0 to 55 wt %, from 5.0 to 50 wt %, from 10 to 45 wt %, from 15 to 42 wt %, from 20 to 41 wt %, from 25 to 40 wt %, from 30 to 39 wt %, from 31 to 38 wt %, or from 32 to 37 wt %. In some embodiments, the sum of the proportions of BaO and ZnO is at least 0.1 wt %, for example at least 1.0 wt %, at least 2.0 wt %, at least 5.0 wt %, at least 10 wt %, at least 15 wt %, at least 20 wt %, at least 25 wt %, at least 30 wt %, at least 31 wt %, or at least 32 wt %. In some embodiments, the sum of the proportions of BaO and ZnO is at most 70 wt %, for example at most 65 wt %, at most 60 wt %, at most 55 wt %, at most 50 wt %, at most 45 wt %, at most 42 wt %, at most 41 wt %, at most 40 wt %, at most 39 wt %, at most 38 wt %, or at most 37 wt %.

In some embodiments, the proportion of TiO₂ is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the proportion of TiO₂ is at least 0.1 wt %, for example at least 0.2 wt %, or at least 0.3 wt %. In some embodiments, the proportion of TiO₂ is at most 5.0 wt %, for example at most 2.0 wt %, at most 1.0 wt %, at most 0.5 wt %, at most 0.2 wt %, at most 0.1 wt %, at most 0.05 wt %, or at most 0.01 wt %. In some embodiments, the glass is free from TiO₂.

In some embodiments, the sum of the proportions by weight of TiO₂, Nb₂O₅ and ZrO₂ is in a range from 0 to 15 wt %, for example from 0.1 to 12 wt %, from 0.2 to 10 wt %, from 0.5 to 8.0 wt %, from 1.0 to 7.0 wt %, from 1.5 to 6.0 wt %, from 2.0 to 5.0 wt %, or from 2.5 to 4.0 wt %. In some embodiments, the sum of the proportions by weight of TiO₂, Nb₂O₅ and ZrO₂ is at least 0.1 wt %, for example at least 0.2 wt %, at least 0.5 wt %, at least 1.0 wt %, at least 1.5 wt %, at least 2.0 wt %, or at least 2.5 wt %. In some embodiments, the sum of the proportions by weight of TiO₂, Nb₂O₅ and ZrO₂ is at most 15 wt %, for example at most 12 wt %, at most 10 wt %, at most 8.0 wt %, at most 7.0 wt %, at most 6.0 wt %, at most 5.0 wt %, or at most 4.0 wt %.

In some embodiments, the ratio of the proportion by weight of Nb₂O₅ to the sum of the proportions by weight of Nb₂O₅, TiO₂ and ZrO₂ is in a range from 0.01 to 0.40, for example from 0.02 to 0.30, from 0.03 to 0.20, or from 0.04 to 0.10. In some embodiments, the ratio of the proportion by weight of Nb₂O₅ to the sum of the proportions by weight of Nb₂O₅, TiO₂ and ZrO₂ is at least 0.01, for example at least 0.02, at least 0.03, or at least 0.04. In some embodiments, the ratio of the proportion by weight of Nb₂O₅ to the sum of the proportions by weight of Nb₂O₅, TiO₂ and ZrO₂ is at most 0.40, for example at most at most 0.30, at most 0.20, at most 0.10, at most 0.07, at most 0.05, at most 0.02, at most 0.01, or even 0.

In some embodiments, the ratio of the proportion by weight of TiO₂ to the proportion by weight of Nb₂O₅ is in a range from 0 to 0.20, for example from 0 to 0.15, from 0 to 0.10, or from 0.01 to 0.05. In some embodiments, the ratio of the proportion by weight of TiO₂ to the proportion by weight of Nb₂O₅ is at least 0.01, for example at least 0.02, or at least 0.05. In some embodiments, the ratio of the proportion by weight of TiO₂ to the proportion by weight of Nb₂O₅ is at most 0.20, for example at most 0.15, at most 0.10, at most 0.05, at most 0.02, at most 0.01, or even 0.

In some embodiments, the sum of the proportions of TiO₂ and Nb₂O₅ is in a range from 0 to 10 wt %, for example from 0 to 9.0 wt %, from 0 to 8.0 wt %, from 0 to 7.0 wt %, from 0.1 to 5.0 wt %, from 0.1 to 2.0 wt %, from 0.1 to 1.5 wt %, from 0.1 to 1.0 wt %, or from 0.1 to 0.5 wt %. In some embodiments, the sum of the proportions of TiO₂ and Nb₂O₅ is at least 0.1 wt %, for example at least 0.2 wt %, or at least 0.5 wt %. In some embodiments, the sum of the proportions of TiO₂ and Nb₂O₅ is at most 10 wt %, for example at most 9.0 wt %, at most 8.0 wt %, at most 7.0 wt %, at most 5.0 wt %, at most 2.0 wt %, at most 1.5 wt %, at most 1.0 wt %, at most 0.5 wt %, at most 0.2 wt %, at most 0.1 wt %, at most 0.05 wt %, or at most 0.01 wt %. In some embodiments, the glass is free from TiO₂ and Nb₂O₅.

In some embodiments, the ratio of the sum of the proportions by weight of TiO₂ and Nb₂O₅ to the proportion by weight of SiO₂ is in a range from 0 to 0.20, for example from 0 to 0.15, from 0 to 0.10, or from 0.01 to 0.05. In some embodiments, the ratio of the sum of the proportions by weight of TiO₂ and Nb₂O₅ to the proportion by weight of SiO₂ is at least 0.01, for example at least 0.02, or at least 0.05. In some embodiments, the ratio of the sum of the proportions by weight of TiO₂ and Nb₂O₅ to the proportion by weight of SiO₂ is at most 0.20, for example at most 0.15, at most 0.10, at most 0.05, at most 0.02, at most 0.01, or even 0.

In some embodiments, the proportion of WO₃ is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the proportion of WO₃ is at least 0.1 wt %, for example at least 0.2 wt %, or at least 0.3 wt %. In some embodiments, the proportion of WO₃ is at most 5.0 wt %, for example at most 2.0 wt %, at most 1.0 wt %, at most 0.5 wt %, at most 0.2 wt %, at most 0.1 wt %, at most 0.05 wt %, or at most 0.01 wt %. In some embodiments, the glass is free from WO₃.

In some embodiments, the ratio of the sum of the proportions by weight of Nb₂O₅ and TiO₂ to the sum of the proportions by weight of Nb₂O₅, TiO₂, Ta₂O₅ and WO₃ is in a range from 0 to 0.30, for example from 0 to 0.25, from 0.01 to 0.20, from 0.02 to 0.10 or from 0.03 to 0.05. In some embodiments, the ratio of the sum of the proportions by weight of Nb₂O₅ and TiO₂ to the sum of the proportions by weight of Nb₂O₅, TiO₂, Ta₂O₅ and WO₃ is at least 0.01, at least 0.02, or at least 0.03. In some embodiments, the ratio of the sum of the proportions by weight of Nb₂O₅ and TiO₂ to the sum of the proportions by weight of Nb₂O₅, TiO₂, Ta₂O₅ and WO₃ is at most 0.30, for example at most 0.25, at most 0.20, at most 0.15, at most 0.10, at most 0.08, at most 0.05, at most 0.02, at most 0.01, or even 0.

In some embodiments, the sum of the proportions of TiO₂, Nb₂O₅ and WO₃ is in a range from 0 to 10 wt %, for example from 0 to 9.0 wt %, from 0 to 8.0 wt %, from 0 to 7.0 wt %, from 0.1 to 5.0 wt %, from 0.1 to 2.0 wt %, from 0.1 to 1.5 wt %, from 0.1 to 1.0 wt %, or from 0.1 to 0.5 wt %. In some embodiments, the sum of the proportions of TiO₂, Nb₂O₅ and WO₃ is at least 0.1 wt %, for example at least 0.2 wt %, or at least 0.5 wt %. In some embodiments, the sum of the proportions of TiO₂, Nb₂O₅ and WO₃ is at most 10 wt %, for example at most 9.0 wt %, at most 8.0 wt %, at most 7.0 wt %, at most 5.0 wt %, at most 2.0 wt %, at most 1.5 wt %, at most 1.0 wt %, at most 0.5 wt %, at most 0.2 wt %, at most 0.1 wt %, at most 0.05 wt %, or at most 0.01 wt %. In some embodiments, the glass is free from TiO₂, Nb₂O₅ and WO₃.

In some embodiments, the sum of the proportions of Nb₂O₅ and WO₃ is in a range from 0 to 10 wt %, for example from 0 to 9.0 wt %, from 0 to 8.0 wt %, from 0 to 7.0 wt %, from 0.1 to 5.0 wt %, from 0.1 to 2.0 wt %, from 0.1 to 1.5 wt %, from 0.1 to 1.0 wt %, or from 0.1 to 0.5 wt %. In some embodiments, the sum of the proportions of Nb₂O₅ and WO₃ is at least 0.1 wt %, for example at least 0.2 wt %, or at least 0.5 wt %. In some embodiments, the sum of the proportions of Nb₂O₅ and WO₃ is at most 10 wt %, for example at most 9.0 wt %, at most 8.0 wt %, at most 7.0 wt %, at most 5.0 wt %, at most 2.0 wt %, at most 1.5 wt %, at most 1.0 wt %, at most 0.5 wt %, at most 0.2 wt %, at most 0.1 wt %, at most 0.05 wt %, or at most 0.01 wt %. In some embodiments, the glass is free from Nb₂O₅ and WO₃.

In some embodiments, the ratio of the proportion by weight of WO₃ to the proportion by weight of ZnO is in a range from 0 to 0.20, for example from 0 to 0.15, from 0 to 0.10, or from 0.01 to 0.05. In some embodiments, the ratio of the proportion by weight of WO₃ to the proportion by weight of ZnO is at least 0.01, for example at least 0.02, or at least 0.05. In some embodiments, the ratio of the proportion by weight of WO₃ to the proportion by weight of ZnO is at most 0.20, for example at most 0.15, at most 0.10, at most 0.05, at most 0.02, at most 0.01, or even 0.

In some embodiments, the ratio of the proportion by weight of WO₃ to the proportion by weight of Ta₂O₅ is in a range from 0 to 0.20, for example from 0 to 0.15, from 0 to 0.10, or from 0.01 to 0.05. In some embodiments, the ratio of the proportion by weight of WO₃ to the proportion by weight of Ta₂O₅ is at least 0.01, for example at least 0.02, or at least 0.05. In some embodiments, the ratio of the proportion by weight of WO₃ to the proportion by weight of Ta₂O₅ is at most 0.20, for example at most 0.15, at most 0.10, at most 0.05, at most 0.02, at most 0.01, or even 0.

In some embodiments, the ratio of the sum of the proportions by weight of WO₃ and TiO₂ to the sum of the proportions by weight of Nb₂O₅ and SiO₂ is in a range from 0 to 0.20, for example from 0 to 0.15, from 0 to 0.10, or from 0.01 to 0.05. In some embodiments, the ratio of the sum of the proportions by weight of WO₃ and TiO₂ to the sum of the proportions by weight of Nb₂O₅ and SiO₂ is at least 0.01, for example at least 0.02, or at least 0.05. In some embodiments, the ratio of the sum of the proportions by weight of WO₃ and TiO₂ to the proportion by weight of Nb₂O₅ and SiO₂ is at most 0.20, for example at most 0.15, at most 0.10, at most 0.05, at most 0.02, at most 0.01, or even 0.

In some embodiments, the proportion of Al₂O₃ is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the proportion of Al₂O₃ is at least 0.1 wt %, for example at least 0.2 wt %, or at least 0.3 wt %. In some embodiments, the proportion of Al₂O₃ is at most 5.0 wt %, for example at most 2.0 wt %, at most 1.0 wt %, at most 0.5 wt %, at most 0.2 wt %, at most 0.1 wt %, at most 0.05 wt %, or at most 0.01 wt %. In some embodiments, the glass is free from Al₂O₃.

In some embodiments, the proportion of Ga₂O₃ is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the proportion of Ga₂O₃ is at least 0.1 wt %, for example at least 0.2 wt %, or at least 0.3 wt %. In some embodiments, the proportion of Ga₂O₃ is at most 5.0 wt %, for example at most 2.0 wt %, at most 1.0 wt %, at most 0.5 wt %, at most 0.2 wt %, at most 0.1 wt %, at most 0.05 wt %, or at most 0.01 wt %. In some embodiments, the glass is free from Ga₂O₃.

In some embodiments, the sum of the proportions of Al₂O₃ and Ga₂O₃ is in a range from 0 to 5.0 wt %, for example from 0 to 2.0 wt %, from 0.1 to 2.0 wt %, from 0 to 1.5 wt %, from 0.2 to 1.5 wt %, from 0 to 1.0 wt %, from 0.3 to 1.0 wt %, from 0 to 0.5 wt %, from 0 to 0.2 wt %, or from 0 to 0.1 wt %. In some embodiments, the sum of the proportions of Al₂O₃ and Ga₂O₃ is at least 0.1 wt %, for example at least 0.2 wt %, or at least 0.3 wt %. In some embodiments, the sum of the proportions of Al₂O₃ and Ga₂O₃ is at most 5.0 wt %, for example at most 2.0 wt %, at most 1.0 wt %, at most 0.5 wt %, at most 0.2 wt %, at most 0.1 wt %, at most 0.05 wt %, or at most 0.01 wt %. In some embodiments, the glass is free from Al₂O₃ and Ga₂O₃.

In some embodiments, the sum of the proportions of B₂O₃, Al₂O₃ and Ga₂O₃ is in a range from 0 to 25 wt %, for example from 0.1 to 20 wt %, from 0.5 to 15 wt %, from 1.0 to 13 wt %, from 2.0 to 10 wt %, from 2.0 to 8.0 wt %, from 2.0 to 6.0 wt %, from 2.0 to 5.0 wt %, from 2.5 to 4.5 wt %, or from 2.5 to 4.0 wt %. In some embodiments, the sum of the proportions of B₂O₃, Al₂O₃ and Ga₂O₃ is at least 0.1 wt %, for example at least 0.2 wt %, at least 0.5 wt %, at least 1.0 wt %, at least 1.5 wt %, at least 2.0 wt %, or at least 2.5 wt %. In some embodiments, the proportion of the sum of the proportions of B₂O₃, Al₂O₃ and Ga₂O₃ is at most 25 wt %, for example at most 20 wt %, at most 15 wt %, at most 13 wt %, at most 10 wt %, at most 8.0 wt %, at most 6.0 wt %, at most 5.0 wt %, at most 4.5 wt %, or at most 4.0 wt %.

In some embodiments, the sum of the proportions by weight of SiO₂ and Al₂O₃ is in a range from 10 to 55 wt %, for example in a range from 15 to 45 wt %, from 17 to 40 wt %, from 18 to 35 wt %, from 20 to 35 wt %, from 22 to 35 wt %, from 23 to 34 wt %, from 24 to 33 wt %, or from 25 to 32 wt %. In some embodiments, the sum of the proportions by weight of SiO₂ and Al₂O₃ is at least 10 wt %, for example at least 15 wt %, at least 17 wt %, at least 18 wt %, at least 20 wt %, at least 22 wt %, at least 23 wt %, at least 24 wt %, or at least 25 wt %. In some embodiments, the sum of the proportions by weight of SiO₂ and Al₂O₃ is at most 55 wt %, at most 45 wt %, at most 40 wt %, at most 35 wt %, at most 34 wt %, at most 33 wt %, or at most 32 wt %.

In some embodiments, the sum of the proportions of Bi₂O₃, La₂O₃, Gd₂O₃, Ta₂O₅, TiO₂, Nb₂O₅ and WO₃ is in a range from 15 to 50 wt %, for example from 20 to 45 wt %, from >20 to 40 wt %, from 21 to 38 wt %, from 22 to 36 wt %, from 23 to 34 wt %, from 24 to 32 wt %, or from 25 to 31 wt %. In some embodiments, the sum of the proportions of Bi₂O₃, La₂O₃, Gd₂O₃, Ta₂O₅, TiO₂, Nb₂O₅ and WO₃ is at least 15 wt %, for example at least 20 wt %, more than 20 wt %, at least 21 wt %, at least 22 wt %, at least 23 wt %, at least 24 wt %, or at least 25 wt %. In some embodiments, the sum of the proportions of Bi₂O₃, La₂O₃, Gd₂O₃, Ta₂O₅, TiO₂, Nb₂O₅ and WO₃ is at most 50 wt %, at most 45 wt %, at most 40 wt %, at most 38 wt %, at most 36 wt %, at most 34 wt %, at most 32 wt %, or at most 31 wt %.

In some embodiments, the sum of the proportions by weight of F, Bi₂O₃, TiO₂, WO₃, Nb₂O₅ and K₂O is in a range from 0 to 10 wt %, for example from 0 to 9.0 wt %, from 0 to 8.0 wt %, from 0 to 7.0 wt %, from 0.1 to 5.0 wt %, from 0.1 to 2.0 wt %, from 0.1 to 1.5 wt %, from 0.1 to 1.0 wt %, or from 0.1 to 0.5 wt %. In some embodiments, the sum of the proportions by weight of F, Bi₂O₃, TiO₂, WO₃, Nb₂O₅ and K₂O is at least 0.1 wt %, for example at least 0.2 wt %, or at least 0.5 wt %. In some embodiments, the sum of the proportions of F, Bi₂O₃, TiO₂, WO₃, Nb₂O₅ and K₂O is at most 10 wt %, for example at most 9.0 wt %, at most 8.0 wt %, at most 7.0 wt %, at most 5.0 wt %, at most 2.0 wt %, at most 1.5 wt %, at most 1.0 wt %, less than 1.0 wt %, at most 0.9 wt %, at most 0.8 wt %, at most 0.7 wt %, at most 0.5 wt %, at most 0.2 wt %, at most 0.1 wt %, at most 0.05 wt %, or at most 0.01 wt %. In some embodiments, the glass is free from F, Bi₂O₃, TiO₂, WO₃, Nb₂O₅ and K₂O.

In some embodiments, the glass contains refining agents selected from Sb₂O₃, As₂O₃, SO₃, SnO₂, Cl and combinations of two or more thereof, in particular in a total proportion of 0.01 to 2.00 wt %, for example from 0.01 to 1.50 wt %, from 0.01 to 1.00 wt %, from 0.01 to 0.75 wt %, from 0.01 to 0.50 wt %, from 0.01 to 0.25 wt %, from 0.01 to 0.20 wt %, from 0.01 to 0.15 wt %, from 0.02 to 0.10 wt %, from 0.02 to 0.08 wt %, from 0.02 to 0.06 wt %, or from 0.03 to 0.05 wt %. In some embodiments, the glass contains refining agents selected from Sb₂O₃, As₂O₃, SO₃, SnO₂, Cl and combinations of two or more thereof, in particular in a total proportion of at least 0.01 wt %, at least 0.02 wt %, or at least 0.03 wt %. In some embodiments, the glass contains refining agents selected from Sb₂O₃, As₂O₃, SO₃, SnO₂, Cl and combinations of two or more thereof, in particular in a total proportion of at most 2.00 wt %, at most 1.50 wt %, at most 1.00 wt %, at most 0.75 wt %, at most 0.50 wt %, at most 0.25 wt %, at most 0.20 wt %, at most 0.15 wt %, at most 0.10 wt %, at most 0.08 wt %, at most 0.06 wt %, or at most 0.05 wt %.

In some embodiments, the proportion of Sb₂O₃ is in a range from 0 to 2.00 wt %, for example from 0 to 1.50 wt %, from 0 to 1.00 wt %, from 0 to 0.75 wt %, from 0 to 0.50 wt %, from 0.01 to 0.25 wt %, from 0.01 to 0.20 wt %, from 0.01 to 0.15 wt %, from 0.02 to 0.10 wt %, from 0.02 to 0.08 wt %, from 0.02 to 0.06 wt %, or from 0.03 to 0.05 wt %. In some embodiments, the proportion of Sb₂O₃ is at least 0.01 wt %, at least 0.02 wt %, or at least 0.03 wt %. In some embodiments, the proportion of Sb₂O₃ is at most 2.00 wt %, at most 1.50 wt %, at most 1.00 wt %, at most 0.75 wt %, at most 0.50 wt %, at most 0.25 wt %, at most 0.20 wt %, at most 0.15 wt %, at most 0.10 wt %, at most 0.08 wt %, at most 0.06 wt %, at most 0.05 wt %, at most 0.04 wt %, at most 0.03 wt %, at most 0.02 wt %, or at most 0.01 wt %. In some embodiments, the glass is free from Sb₂O₃.

In some embodiments, the proportion of As₂O₃ is at most 1.0 wt %, for example at most 0.5 wt %, at most 0.2 wt %, at most 0.1 wt %, at most 0.05 wt %, or at most 0.01 wt %. In some embodiments, the glass is free from As₂O₃.

In some embodiments, the proportion of SO₃ is at most 1.0 wt %, for example at most 0.5 wt %, at most 0.2 wt %, at most 0.1 wt %, at most 0.05 wt %, or at most 0.01 wt %. In some embodiments, the glass is free from SO₃.

In some embodiments, the total proportion of Sn oxides, in particular the total proportion of SnO₂ and SnO, is at most 1.0 wt %, for example at most 0.5 wt %, at most 0.2 wt %, at most 0.1 wt %, at most 0.05 wt %, or at most 0.01 wt %. In some embodiment, the glass is free from Sn oxides, in particular free from SnO₂ and SnO.

In some embodiments, the proportion of Cl is at most 1.0 wt %, for example at most 0.5 wt %, at most 0.2 wt %, at most 0.1 wt %, at most 0.05 wt %, or at most 0.01 wt %. In some embodiments, the glass is free from Cl.

In some embodiments, the proportion of Ag₂O is at most 1.0 wt %, for example at most 0.5 wt %, at most 0.2 wt %, at most 0.1 wt %, at most 0.05 wt %, or at most 0.01 wt %. In some embodiments, the glass is free from Ag₂O.

In some embodiments, the proportion of at least one of Cr₂O₃, NiO, Fe₂O₃ and Pt is at most 1.0 wt %, for example at most 0.5 wt %, at most 0.2 wt %, at most 0.1 wt %, at most 0.05 wt %, or at most 0.01 wt %. In some embodiments, the proportion of Cr₂O₃, NiO, Fe₂O₃ and Pt is in each case at most 1.0 wt %, for example at most 0.5 wt %, at most 0.2 wt %, at most 0.1 wt %, at most 0.05 wt %, or at most 0.01 wt %. In some embodiments, the sum of the proportions of Cr₂O₃, NiO, Fe₂O₃ and Pt is at most 1.0 wt %, for example at most 0.5 wt %, at most 0.2 wt %, at most 0.1 wt %, at most 0.05 wt %, or at most 0.01 wt %. In some embodiments, the glass is free from at least one of Cr₂O₃, NiO, Fe₂O₃ and Pt. In some embodiments, the glass is free from Cr₂O₃, NiO, Fe₂O₃ and Pt.

In some embodiments, the proportion of Bi₂O₃ is at most 1.0 wt %, for example at most 0.5 wt %, at most 0.2 wt %, at most 0.1 wt %, at most 0.05 wt %, or at most 0.01 wt %. In some embodiments, the glass is free from Bi₂O₃.

In some embodiments, the proportion of Tb₂O₃ is at most 1.0 wt %, for example at most 0.5 wt %, at most 0.2 wt %, at most 0.1 wt %, at most 0.05 wt %, or at most 0.01 wt %. In some embodiments, the glass is free from Tb₂O₃.

In some embodiments, the proportion of Eu₂O₃ is at most 1.0 wt %, for example at most 0.5 wt %, at most 0.2 wt %, at most 0.1 wt %, at most 0.05 wt %, or at most 0.01 wt %. In some embodiments, the glass is free from Eu₂O₃.

In some embodiments, the proportion of at least one of F, Cl and I is at most 1.0 wt %, for example at most 0.5 wt %, at most 0.2 wt %, at most 0.1 wt %, at most 0.05 wt %, or at most 0.01 wt %. In some embodiments, the proportion of F, Cl and I is in each case at most 1.0 wt %, for example at most 0.5 wt %, at most 0.2 wt %, at most 0.1 wt %, at most 0.05 wt %, or at most 0.01 wt %. In some embodiments, the sum of the proportions of F, Cl and I is at most 1.0 wt %, for example at most 0.5 wt %, at most 0.2 wt %, at most 0.1 wt %, at most 0.05 wt %, or at most 0.01 wt %. In some embodiments, the glass is free from at least one of F, Cl and I. In some embodiments, the glass is free from F, Cl and I.

In some embodiments, the proportion of GeO₂ is at most 1.0 wt %, for example at most 0.5 wt %, at most 0.2 wt %, at most 0.1 wt %, at most 0.05 wt %, or at most 0.01 wt %. In some embodiments, the glass is free from GeO₂.

In some embodiments, the proportion of P₂O₅ is at most 1.0 wt %, for example at most 0.5 wt %, at most 0.2 wt %, at most 0.1 wt %, at most 0.05 wt %, or at most 0.01 wt %. In some embodiments, the glass is free from P₂O₅.

In some embodiments, the sum of the proportions of Al₂O₃, GeO₂, Ga₂O₃ and P₂O₅ is at most 1.0 wt %, for example at most 0.5 wt %, at most 0.2 wt %, at most 0.1 wt %, at most 0.05 wt %, or at most 0.01 wt %. In some embodiments, the glass is free from Al₂O₃, GeO₂, Ga₂O₃ and P₂O₅.

In some embodiments, the sum of the proportions of GeO₂ and Ga₂O₃ is at most 1.0 wt %, for example at most 0.5 wt %, at most 0.2 wt %, at most 0.1 wt %, at most 0.05 wt %, or at most 0.01 wt %. In some embodiments, the glass is free from GeO₂ and Ga₂O₃.

In some embodiments, the proportion of CuO is at most 1.0 wt %, for example at most 0.5 wt %, at most 0.2 wt %, at most 0.1 wt %, at most 0.05 wt %, or at most 0.01 wt %. In some embodiments, the glass is free from CuO.

In some embodiments, the sum of the proportions of V, Cr, Mn, Fe, Co, Ni, Cu, Mo, Ru, Ce, Pr and Er oxides is at most 1.0 wt %, for example at most 0.5 wt %, at most 0.2 wt %, at most 0.1 wt %, at most 0.05 wt %, or at most 0.01 wt %. In some embodiments, the glass is free from V, Cr, Mn, Fe, Co, Ni, Cu, Mo, Ru, Ce, Pr and Er oxides.

In some embodiments, the sum of the proportions of CeO₂ and Tb₂O₃ is at most 1.0 wt %, for example at most 0.5 wt %, at most 0.2 wt %, at most 0.1 wt %, at most 0.05 wt %, or at most 0.01 wt %. In some embodiments, the glass is free from CeO₂ and Tb₂O₃.

In some embodiments, the proportion of CeO₂ is at most 1.0 wt %, for example at most 0.5 wt %, at most 0.2 wt %, at most 0.1 wt %, at most 0.05 wt %, or at most 0.01 wt %. In some embodiments, the glass is free from CeO₂.

In some embodiments, the total proportion of sulfur oxides, in particular the total proportion of SO₂ and SO₃, is at most 1.0 wt %, for example at most 0.5 wt %, at most 0.2 wt %, at most 0.1 wt %, at most 0.05 wt %, or at most 0.01 wt %. In some embodiments, the glass is sulfur-free, in particular is free from SO₂ and SO₃.

In some embodiments, the proportion of at least one of As₂O₃ and PbO is at most 1.0 wt %, for example at most 0.5 wt %, at most 0.2 wt %, at most 0.1 wt %, at most 0.05 wt %, or at most 0.01 wt %. In some embodiments, the proportion of As₂O₃ and PbO is in each case at most 1.0 wt %, for example at most 0.5 wt %, at most 0.2 wt %, at most 0.1 wt %, at most 0.05 wt %, or at most 0.01 wt %. In some embodiments, the sum of the proportions of As₂O₃ and PbO is at most 1.0 wt %, for example at most 0.5 wt %, at most 0.2 wt %, at most 0.1 wt %, at most 0.05 wt %, or at most 0.01 wt %. In some embodiments, the glass is free from at least one of As₂O₃ and PbO. In some embodiments, the glass is free from As₂O₃ and PbO.

When it is stated in this disclosure that the glass is “free from” a constituent or that it does not contain a certain constituent, this means that this constituent may still be present as an impurity in the glass. This means that it is not added in substantial quantities. Insubstantial quantities are quantities of less than 100 ppm (proportion by weight), less than 75 ppm (proportion by weight), less than 50 ppm (proportion by weight), less than 25 ppm (proportion by weight), in particular less than 10 ppm (proportion by weight).

In some preferred embodiments, the glass comprises the following components in the stated proportions (by weight):

| Component | Min (wt %) | Max (wt %) |
|---|---|---|
| SiO₂ | 10 | 55 |
| B₂O₃ | 0 | 25 |
| CaO | 0 | 5.0 |
| BaO | 0 | 50 |
| SrO | 0 | 5.0 |
| ZnO | 0 | 30 |
| La₂O₃ | 0 | 70 |
| Gd₂O₃ | 0 | 15 |
| Y₂O₃ | 0 | 15 |
| ZrO₂ | 0.1 | 10 |
| Ta₂O₅ | 0 | 10 |
| Nb₂O₅ | 0 | 10 |
| Σ R₂O | 0 | 10 |

In more preferred embodiments, the glass comprises the following components in the stated proportions (by weight):

| Component | Min (wt %) | Max (wt %) |
|---|---|---|
| SiO₂ | 15 | 45 |
| B₂O₃ | 0.1 | 20 |
| CaO | 0 | 2.0 |
| BaO | 0.1 | 45 |
| SrO | 0 | 3.0 |
| ZnO | 0.1 | 25 |
| La₂O₃ | 0.1 | 50 |
| Gd₂O₃ | 0 | 10 |
| Y₂O₃ | 0 | 10 |
| ZrO₂ | 0.2 | 9.0 |
| Ta₂O₅ | 0 | 9.0 |
| Nb₂O₅ | 0 | 9.0 |
| Σ R₂O | 0 | 4.0 |

In more preferred embodiments, the glass comprises the following components in the stated proportions (by weight):

| Component | Min (wt %) | Max (wt %) |
|---|---|---|
| SiO₂ | 17 | 40 |
| B₂O₃ | 0.5 | 15 |
| CaO | 0 | 1.0 |
| BaO | 1.0 | 35 |
| SrO | 0 | 2.5 |
| ZnO | 0.5 | 20 |
| La₂O₃ | 2.0 | 45 |
| Gd₂O₃ | 0 | 9.0 |
| Y₂O₃ | 0 | 9.0 |
| ZrO₂ | 0.5 | 8.0 |
| Ta₂O₅ | 0 | 8.0 |
| Nb₂O₅ | 0 | 8.0 |
| Σ R₂O | 0 | 3.0 |

In more preferred embodiments, the glass comprises the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

In more preferred embodiments, the glass comprises the following components in the stated proportions (by weight):

| Component | Min (wt %) | Max (wt %) |
|---|---|---|
| SiO₂ | 20 | 35 |
| B₂O₃ | 2.0 | 10 |
| CaO | 0 | 0.2 |
| BaO | 5.0 | 30 |
| SrO | 0 | 1.0 |
| ZnO | 2.0 | 18 |
| La₂O₃ | 10 | 30 |
| Gd₂O₃ | 0.1 | 7.0 |
| Y₂O₃ | 0.1 | 7.0 |
| ZrO₂ | 1.0 | 5.0 |
| Ta₂O₅ | 0.1 | 7.0 |
| Nb₂O₅ | 0.1 | 5.0 |
| Σ R₂O | 0.1 | 1.5 |

In more preferred embodiments, the glass comprises the following components in the stated proportions (by weight):

| Component | Min (wt %) | Max (wt %) |
|---|---|---|
| SiO₂ | 22 | 35 |
| B₂O₃ | 2.0 | 8.0 |
| CaO | 0 | 0.1 |
| BaO | 10 | 30 |
| SrO | 0 | 0.5 |
| ZnO | 5.0 | 18 |
| La₂O₃ | 15 | 25 |
| Gd₂O₃ | 0.2 | 7.0 |
| Y₂O₃ | 0.2 | 5.0 |
| ZrO₂ | 1.5 | 4.5 |
| Ta₂O₅ | 0.2 | 7.0 |
| Nb₂O₅ | 0.1 | 2.0 |
| Σ R₂O | 0.1 | 1.2 |

In more preferred embodiments, the glass comprises the following components in the stated proportions (by weight):

| Component | Min (wt %) | Max (wt %) |
|---|---|---|
| SiO₂ | 22 | 35 |
| B₂O₃ | 2.0 | 8.0 |
| CaO | 0 | 0.1 |
| BaO | 15 | 30 |
| SrO | 0 | 0 |
| ZnO | 2.0 | 18 |
| La₂O₃ | 10 | 30 |
| Gd₂O₃ | 0.5 | 7.0 |
| Y₂O₃ | 1.0 | 10 |
| ZrO₂ | 1.0 | 5.0 |
| Ta₂O₅ | 1.0 | 7.0 |
| Nb₂O₅ | 0 | 0 |
| Σ R₂O | 0 | 2.0 |

In more preferred embodiments, the glass comprises the following components in the stated proportions (by weight):

| Component | Min (wt %) | Max (wt %) |
|---|---|---|
| SiO₂ | 23 | 34 |
| B₂O₃ | 2.0 | 6.0 |
| CaO | 0 | 0.1 |
| BaO | 15 | 28 |
| SrO | 0 | 0.5 |
| ZnO | 10 | 18 |
| La₂O₃ | 15 | 25 |
| Gd₂O₃ | 0.5 | 7.0 |
| Y₂O₃ | 0.5 | 2.5 |
| ZrO₂ | 1.5 | 4.5 |
| Ta₂O₅ | 0.5 | 7.0 |
| Nb₂O₅ | 0.1 | 1.5 |
| Σ R₂O | 0.1 | 1.1 |

In more preferred embodiments, the glass comprises the following components in the stated proportions (by weight):

| Component | Min (wt %) | Max (wt %) |
|---|---|---|
| SiO₂ | 24 | 33 |
| B₂O₃ | 2.0 | 5.0 |
| CaO | 0 | 0.1 |
| BaO | 17 | 27 |
| SrO | 0 | 0.5 |
| ZnO | 11 | 17 |
| La₂O₃ | 15 | 25 |
| Gd₂O₃ | 1.0 | 7.0 |
| Y₂O₃ | 0.5 | 2.0 |
| ZrO₂ | 2.0 | 4.5 |
| Ta₂O₅ | 1.0 | 7.0 |
| Nb₂O₅ | 0.1 | 1.0 |
| Σ R₂O | 0.1 | 1.1 |

In more preferred embodiments, the glass comprises the following components in the stated proportions (by weight):

| Component | Min (wt %) | Max (wt %) |
|---|---|---|
| SiO₂ | 24 | 32 |
| B₂O₃ | 2.0 | 5.0 |
| CaO | 0 | 0.1 |
| BaO | 17 | 26 |
| SrO | 0 | 0 |
| ZnO | 5.0 | 15 |
| La₂O₃ | 17 | 28 |
| Gd₂O₃ | 1.5 | 6.0 |
| Y₂O₃ | 2.0 | 8.0 |
| ZrO₂ | 2.0 | 4.5 |
| Ta₂O₅ | 2.0 | 7.0 |
| Nb₂O₅ | 0 | 0 |
| Σ R₂O | 0 | 2.0 |

In more preferred embodiments, the glass comprises the following components in the stated proportions (by weight):

| Component | Min (wt %) | Max (wt %) |
|---|---|---|
| SiO₂ | 25 | 32 |
| B₂O₃ | 2.5 | 4.5 |
| CaO | 0 | 0.1 |
| BaO | 17 | 26 |
| SrO | 0 | 0.5 |
| ZnO | 11 | 16 |
| La₂O₃ | 15 | 24 |
| Gd₂O₃ | 1.5 | 6.0 |
| Y₂O₃ | 0.5 | 1.5 |
| ZrO₂ | 2.0 | 4.5 |
| Ta₂O₅ | 2.0 | 7.0 |
| Nb₂O₅ | 0.1 | 0.5 |
| Σ R₂O | 0.2 | 1.1 |

In more preferred embodiments, the glass comprises the following components in the stated proportions (by weight):

| Component | Min (wt %) | Max (wt %) |
|---|---|---|
| SiO₂ | 27 | 31 |
| B₂O₃ | 3.0 | 4.0 |
| CaO | 0 | 0 |
| BaO | 17 | 21 |
| SrO | 0 | 0 |
| ZnO | 7.0 | 14 |
| La₂O₃ | 19 | 26 |
| Gd₂O₃ | 3.0 | 5.5 |
| Y₂O₃ | 2.5 | 6.0 |
| ZrO₂ | 2.0 | 3.5 |
| Ta₂O₅ | 2.0 | 5.0 |
| Nb₂O₅ | 0 | 0 |
| Σ R₂O | 0 | 2.0 |

### Optical Properties

In some embodiments, for a sample thickness of 25 mm and a wavelength of 380 nm, the glass has a pure transmittance of at least 0.900, for example a pure transmittance of at least 0.910, at least 0.920, at least 0.930, at least 0.940, at least 0.950, at least 0.960, at least 0.965, at least 0.970, at least 0.975 or at least 0.980. In some embodiments, for a sample thickness of 25 mm and a wavelength of 380 nm, the glass has a pure transmittance of at most 0.999, at most 0.998, at most 0.995, at most 0.990, or at most 0.985. In some embodiments, for a sample thickness of 25 mm and a wavelength of 380 nm, the glass has a pure transmittance in a range from 0.900 to 0.999, for example in a range from 0.910 to 0.999, from 0.920 to 0.998, from 0.930 to 0.998, from 0.940 to 0.995, from 0.950 to 0.995, from 0.960 to 0.995, from 0.965 to 0.990, from 0.970 to 0.990, from 0.975 to 0.985, or from 0.980 to 0.985.

In some embodiments, for a sample thickness of 25 mm and in the overall wavelength range from 380 nm to 700 nm, the glass has a pure transmittance of at least 0.900, for example a pure transmittance of at least 0.910, at least 0.920, at least 0.930, at least 0.940, at least 0.950, at least 0.960, at least 0.965, at least 0.970, at least 0.975 or at least 0.980. In some embodiments, for a sample thickness of 25 mm and in the overall wavelength range from 380 nm to 700 nm, the glass has a pure transmittance of at most 0.999, at most 0.998, at most 0.995, at most 0.990, or at most 0.985. In some embodiments, for a sample thickness of 25 mm and in the overall wavelength range from 380 nm to 700 nm, the glass has a pure transmittance in a range from 0.900 to 0.999, for example in a range from 0.910 to 0.999, from 0.920 to 0.998, from 0.930 to 0.998, from 0.940 to 0.995, from 0.950 to 0.995, from 0.960 to 0.995, from 0.965 to 0.990, from 0.970 to 0.990, from 0.975 to 0.985, or from 0.980 to 0.985.

In some embodiments, for a wavelength of 380 nm, the glass has an attenuation in a range from 0.5 to <10 dB/m, for example from 1.0 to <5.0 dB/m, from 1.5 to 4.0 dB/m, or from 2.0 to 3.5 dB/m. In some embodiments, for a wavelength of 380 nm, the glass has an attenuation or at least 0.5 dB/m, for example at least 1.0 dB/m, at least 1.5 dB/m, or at least 2.0 dB/m. In some embodiments, for a wavelength of 380 nm, the glass has an attenuation of less than 10 dB/m, for example less than 5.0 dB/m, at most 4.0 dB/m, or at most 3.5 dB/m.

In some embodiments, for a sample thickness in each case of 25 mm, the ratio of the pure transmittance for a wavelength of 380 nm to the pure transmittance for a wavelength of 600 nm is at least 0.900, for example at least 0.910, at least 0.920, at least 0.930, at least 0.940, at least 0.950, at least 0.960, at least 0.965, at least 0.970, at least 0.975, or at least 0.980. In some embodiments, for a sample thickness in each case of 25 mm, the ratio of the pure transmittance for a wavelength of 380 nm to the pure transmittance for a wavelength of 600 nm is at most 0.999, at most 0.998, at most 0.995, at most 0.990, or at most 0.985. In some embodiments, for a sample thickness in each case of 25 mm, the ratio of the pure transmittance for a wavelength of 380 nm to the pure transmittance for a wavelength of 600 nm in a range from 0.900 to 0.999, for example in a range from 0.910 to 0.999, from 0.920 to 0.998, from 0.930 to 0.998, from 0.940 to 0.995, from 0.950 to 0.995, from 0.960 to 0.995, from 0.965 to 0.990, from 0.970 to 0.990, from 0.975 to 0.985, or from 0.980 to 0.985.

In some embodiments, the refractive index n_{d} is in a range from 1.60 to 1.85, for example in a range from 1.65 to 1.80, from 1.67 to 1.77, from 1.68 to 1.75, from 1.69 to 1.74, or 1.70 to 1.73. In some embodiments, the refractive index n_{d} is at least 1.60, for example at least 1.65, at least 1.67, at least 1.68, at least 1.69, or at least 1.70. In some embodiments, the refractive index n_{d} is at most 1.85, for example at most 1.80, at most 1.77, at most 1.75, at most 1.74, or at most 1.73.

In some embodiments, the refractive index v_{d} is in a range from 35 to 60, for example from 40 to 55, from 42 to 54, from 43 to 53, from 44 to 52, or from 45 to 51. In some embodiments, the Abbe number vd is at least 35, for example at least 40, at least 42, at least 43, at least 44, or at least 45. In some embodiments, the Abbe number vd is at most 60, for example at most 55, at most 54, at most 53, at most 52, or at most 51.

### Resistance to Crystallization

Not only do the glasses of the invention have outstanding optical properties, the glasses of the invention are also characterized in that they have particularly high resistance to crystallization. This is of particular importance for production in fibre drawing. Otherwise, devitrification crystals can form, in particular on the surface of the glass, resulting for example in impaired shaping of the glass. Therefore, the glasses have good devitrification stability. This is particularly important for large dimensions and thick walls.

One measure of resistance to crystallization is the maximum rate of crystallization KGₘₐₓ. The lower KGₘₐₓ, the greater the resistance to crystallization. In the present disclosure, the terms “resistance to crystallization” and “devitrification stability” are used synonymously. KGₘₐₓ describes the maximum rate of crystallization (generally in μm/min). Measurement of the rate of crystallization is known. The rate of crystallization is preferably measured along formed crystals, i.e. along the longest extension thereof.

According to the invention, “LDP” means the “lower devitrification point”. This is the temperature at which, with rising temperature control, the devitrification of the material begins. Above a certain temperature, denoted as upper devitrification point (UDP) or liquidus temperature, no crystals are formed even after long periods. The numerical values of LDP and UDP generally differ in different glasses.

If crystallization occurs, it does so at temperatures above the lower devitrification point (LDP) and below the upper devitrification point (UDP), i.e. in a range between LDP and UDP. The temperature at which the maximum rate of crystallization is achieved is therefore also between LDP and UDP. In order to determine the maximum rate of crystallization KGₘₐₓ, therefore, the glass has to be heated to a temperature between LDP and UDP. Because it is not known for a given glass where precisely the maximum crystallization is in the range between LDP and UDP, different temperatures within that range are commonly tested in order to determine KGₘₐₓ. In this way, it is also possible for LDP and UDP themselves to be determined to be the lower and upper limits of the range in which crystallization takes place.

When reference is made in the present disclosure to the lower devitrification point LDP, this therefore means the LDP which was determined by thermally treating the glass for a retention time of 5 minutes in a gradient furnace with rising temperature control, unless otherwise indicated.

In particular, the rate of crystallization is determined by thermally treating the glass for a retention time of 5 minutes or one hour in a gradient furnace with rising temperature control. A gradient furnace is a furnace with different heating zones, i.e. a furnace with different temperature regions. Rising temperature control means that the temperature of the glass before it is introduced into the gradient furnace is lower than the temperatures in all the regions of the furnace. The temperature of the glass therefore increases by being introduced into the furnace, regardless of which region of the furnace the glass is introduced into. The devitrification measurement is therefore carried out in particular for a five-minute or one-hour thermal treatment in an (already hot) gradient furnace split into different temperature zones. The temperature gradient in the gradient furnace is spatially resolved rather than temporally resolved, since the gradient furnace is spatially split into different temperature zones.

Because the gradient furnace is split into a plurality of heating zones, different temperatures can be tested at the same time. This is a particular advantage of a gradient furnace. For example, the lowest temperature can be 950° C. and the highest temperature 1250° C., or the lowest temperature can be 700° C. and the highest temperature 1000° C. The temperatures should be chosen such that the rate of crystallization at different temperatures in the range between LDP and UDP can be determined such that, by comparing the potentially different rates of crystallization within the range between LDP and UDP, the highest rate of crystallization can be determined to be the maximum rate of crystallization KGₘₐₓ. If LDP and UDP are unknown, a relatively wide range of temperatures are tested in order to enable LDP and UDP to be determined.

The fact that a glass has, for example, a maximum rate of crystallization (KGₘₐₓ) of at most 15 μm/min in a temperature range from 700° C. to 1250° C. when the glass is thermally treated for a retention time of one hour in a gradient furnace with rising temperature control, therefore does not mean that temperatures over the whole range from 700° C. to 1250° C. have to be present in the gradient furnace. For example, if it is known for a certain glass that the UDP is 1000° C., no temperatures greater than 1000° C. have to be tested in the gradient furnace, since crystallization no longer occurs at these temperatures anyway, and therefore the maximum rate of crystallization KGₘₐₓ must be below 1000° C. The same applies, that no temperatures below 950° C. have to be tested in the gradient furnace, for example, if it is known for a glass that LDP is 950° C., since crystallization does not occur at these temperatures anyway, and therefore the maximum rate of crystallization KGₘₐₓ must be above 950° C.

If no devitrification occurs, there is no crystallization, and therefore KGₘₐₓ cannot be determined. In this case, a value of 0 μm/min can be assumed for KGₘₐₓ.

The rate of crystallization is preferably determined using glass grain, in particular glass grain having a diameter of 1.6 mm to 4 mm. For the thermal treatment in the gradient furnace, glass grain is preferably placed on a support, for example a platinum support. The support can have depressions, in particular for receiving one grain of glass each, and a hole on the underside of each depression, such that the rate of crystallization following the thermal treatment can be determined microscopically. Regarding the preferred size of the grains of glass, the depressions preferably each have a diameter of 4 mm and the holes each have a diameter of 1 mm. Following the thermal treatment it is possible to microscopically determine which rate of crystallization was present in which temperature range. The highest rate of crystallization ascertained is the maximum rate of crystallization KGₘₐₓ. LDP and UDP can be determined as the lower and upper limits of the temperature range in which crystallization took place. Assigning the individual grains of glass to the different temperature zones in the gradient furnace is unproblematic, since it is known what temperature is present at what position in the furnace, and which glass grain was located at what position in the furnace.

The glasses of the invention have such a high devitrification stability that in some embodiments the maximum rate of crystallization (KGₘₐₓ) is at most 7.5 μm/min in a temperature range from 700° C. to 1250° C. (in particular 800° C. to 1200° C., 850° C. to 1150° C., 900° C. to 1100° C., or 950° C. to 1050° C.) when the glass is thermally treated for a retention time of one hour in a gradient furnace with rising temperature control. In some embodiments, KGₘₐₓ is at most 6.0 μm/min in a temperature range from 700° C. to 1250° C. (in particular 800° C. to 1200° C., 850° C. to 1150° C., 900° C. to 1100° C., or 950° C. to 1050° C.), for example at most 5.0 μm/min, at most 4.0 μm/min, at most 3.0 μm/min, at most 2.5 μm/min, at most 2.0 μm/min, at most 1.5 μm/min, at most 1.0 μm/min, at most 0.5 μm/min, at most 0.2 μm/min, at most 0.1 μm/min, or even 0 μm/min, when the glass is thermally treated for a retention time of one hour in a gradient furnace with rising temperature control. In some embodiments, KGₘₐₓ is at least 0.1 μm/min in a temperature range from 700° C. to 1250° C. (in particular 800° C. to 1200° C., 850° C. to 1150° C., 900° C. to 1100° C., or 950° C. to 1050° C.), for example at least 0.2 μm/min, at least 0.5 μm/min, at least 1.0 μm/min, at least 1.5 μm/min, at least 2.0 μm/min, at least 2.5 μm/min, or at least 3.0 μm/min, when the glass is thermally treated for a retention time of one hour in a gradient furnace with rising temperature control. In some embodiments, in a temperature range from 700° C. to 1250° C. (in particular 800° C. to 1200° C., 850° C. to 1150° C., 900° C. to 1100° C., or 950° C. to 1050° C.) KGₘₐₓ is in a range from 0 to 7.5 μm/min, for example in a range from 0 to 6.0 μm/min, from 0.1 to 5.0 μm, from 0.2 to 4.0 μm/min, from 0.5 to 3.0 μm/min, from 1.0 to 2.5 μm/min, from 1.5 to 2.0 μm/min, from 1.0 to 1.5 μm/min, from 0.5 to 1.0 μm/min, from 0.2 to 0.5 μm/min, from 0.1 to 0.2 μm/min, or from 0 to 0.1 μm/min, when the glass is thermally treated for a retention time of one hour in a gradient furnace with rising temperature control.

In some embodiments, UDP is in a range from 900° C. to 1400° C., for example from 950° C. to 1350° C., from 1000° C. to 1300° C., from 1050° C. to 1250° C., or from 1100° C. to 1200° C., when the glass is thermally treated for a retention time of one hour in a gradient furnace with rising temperature control. In some embodiments, UDP is at least 900° C., for example at least 950° C., at least 1000° C., at least 1050° C., or at least 1100° C., when the glass is thermally treated for a retention time of one hour in a gradient furnace with rising temperature control. In some embodiments, UDP is at most 1400° C., for example at most 1350° C., at most 1300° C., at most 1250° C., or at most 1200° C., when the glass is thermally treated for a retention time of one hour in a gradient furnace with rising temperature control.

The glasses of the invention have such a high devitrification stability that in some embodiments the maximum rate of crystallization (KGₘₐₓ) is at most 25 μm/min in a temperature range from 700° C. to 1250° C. (in particular 850° C. to 1200° C., 900° C. to 1150° C., 950° C. to 1100° C., or 1000° C. to 1050° C.) when the glass is thermally treated for a retention time of 5 minutes in a gradient furnace with rising temperature control. In some embodiments, KGₘₐₓ is at most 20 μm/min in a temperature range from 700° C. to 1250° C. (in particular 850° C. to 1200° C., 900° C. to 1150° C., 950° C. to 1100° C., or 1000° C. to 1050° C.), for example at most 15 μm/min, at most 12 μm/min, at most 10 μm/min, at most 7.5 μm/min, at most 5.0 μm/min, at most 4.0 μm/min, at most 3.0 μm/min, at most 2.0 μm/min, at most 1.0 μm/min, at most 0.5 μm/min, at most 0.2 μm/min, at most 0.1 μm/min, or even 0 μm/min, when the glass is thermally treated for a retention time of 5 minutes in a gradient furnace with rising temperature control. In some embodiments, KGₘₐₓ is at least 0.1 μm/min in a temperature range from 700° C. to 1250° C. (in particular 850° C. to 1200° C., 900° C. to 1150° C., 950° C. to 1100° C., or 1000° C. to 1050° C.), for example at least 0.2 μm/min, at least 0.5 μm/min, at least 1.0 μm/min, at least 2.0 μm/min, at least 3.0 μm/min, at least 5.0 μm/min, or at least 7.5 μm/min, when the glass is thermally treated for a retention time of 5 minutes in a gradient furnace with rising temperature control. In some embodiments, in a temperature range from 700° C. to 1250° C. (in particular 850° C. to 1200° C., 900° C. to 1150° C., 950° C. to 1100° C., or 1000° C. to 1050° C.) KGₘₐₓ is in a range from 0 to 25 μm/min, for example in a range from 0.1 to 20 μm/min, from 0.5 to 15 μm, from 1.0 to 12 μm/min, from 2.0 to 10 μm/min, from 3.0 to 7.5 μm/min, from 2.0 to 5.0 μm/min, from 1.0 to 4.0 μm/min, from 0.5 to 3.0 μm/min, from 0.2 to 2.0 μm/min, from 0.1 to 1.0 μm/min, or from 0 to 0.1 μm/min, when the glass is thermally treated for a retention time of 5 minutes in a gradient furnace with rising temperature control.

In some embodiments, LDP is in a range from 650° C. to 1100° C., for example from 700° C. to 1050° C., from 750° C. to 1000° C., from 800° C. to 950° C., or from 850° C. to 900° C., when the glass is thermally treated for a retention time of 5 minutes in a gradient furnace with rising temperature control. In some embodiments, LDP is at least 650° C., for example at least 700° C., at least 750° C., at least 800° C., or at least 850° C., when the glass is thermally treated for a retention time of 5 minutes in a gradient furnace with rising temperature control. In some embodiments, LDP is at most 1100° C., for example at most 1050° C., at most 1000° C., at most 950° C., or at most 900° C., when the glass is thermally treated for a retention time of 5 minutes in a gradient furnace with rising temperature control.

In some embodiments, UDP is in a range from 850° C. to 1350° C., for example from 900° C. to 1300° C., from 950° C. to 1250° C., from 1000° C. to 1200° C., or from 1050° C. to 1150° C., when the glass is thermally treated for a retention time of 5 minutes in a gradient furnace with rising temperature control. In some embodiments, UDP is at least 850° C., for example at least 900° C., at least 950° C., at least 1000° C., or at least 1050° C., when the glass is thermally treated for a retention time of 5 minutes in a gradient furnace with rising temperature control. In some embodiments, UDP is at most 1350° C., for example at most 1300° C., at most 1250° C., at most 1200° C., or at most 1150° C., when the glass is thermally treated for a retention time of 5 minutes in a gradient furnace with rising temperature control.

In some embodiments, the difference between UDP and LDP is in a range from 100 to 300 K, for example in a range from 125 to 275 K, from 150 to 250 K, or from 175 to 225 K, when the glass is thermally treated for a retention time of 5 minutes in a gradient furnace with rising temperature control. In some embodiments, the difference between UDP and LDP is at least 100 K, for example at least at least 125 K, at least 150 K, or at least 175 K, when the glass is thermally treated for a retention time of 5 minutes in a gradient furnace with rising temperature control. In some embodiments, the difference between UDP and LDP is at most 300 K, for example at most at most 275 K, at most 250 K, or at most 225 K, when the glass is thermally treated for a retention time of 5 minutes in a gradient furnace with rising temperature control.

By virtue of its outstanding devitrification stability, the glass of the invention can be produced in any known glass shaping method, in particular tube drawing methods, for example in the Danner method, in the Vello method or in the vertical drawing method.

### Further Properties

The annealing point T13 is the temperature at which the viscosity is 10¹³ dPas. In some embodiments, the annealing point T13 is in a range from 550° C. to 750° C., for example from 575° C. to 725° C., from 600° C. to 700° C., or from 625° C. to 675° C. In some embodiments, the annealing point T13 is at least 550° C., for example at least 575° C., at least 600° C., or at least 625° C. In some embodiments, the annealing point T13 is at most 750° C., for example at most 725° C., at most 700° C., or at most 675° C.

The softening point T7.6 is the temperature at which the viscosity is 10^{7.6} dPas. In some embodiments, the softening point T7.6 is in a range from 700° C. to 900° C., for example from 725° C. to 875° C., from 750° C. to 850° C., or from 775° C. to 825° C. In some embodiments, the softening point T7.6 is at least 700° C., for example at least 725° C., at least 750° C., or at least 775° C. In some embodiments, the softening point T7.6 is at most 900° C., for example at most 875° C., at most 850° C., or at most 825° C.

In some embodiments, the difference between the lower devitrification point LDP and the softening point T7.6 is in a range from 25 to 175 K, for example from 40 to 160 K, from 50 to 150 K, from 60 to 140 K, from 70 to 130 K, or from 75 to 125 K. In some embodiments, the difference between the lower devitrification point LDP and the softening point T7.6 is at least 25 K, for example at least 40 K, at least 50 K, at least 60 K, at least 70 K, or at least 75 K. In some embodiments, the difference between the lower devitrification point LDP and the softening point T7.6 is at most 175 K, for example at most 160 K, at most 150 K, at most 140 K, at most 130 K, or at most 125 K.

The working point T4 is the temperature at which the viscosity is 104 dPas. In some embodiments, the working point T4 is in a range from 800° C. to 1100° C., for example from 850° C. to 1050° C., from 875° C. to 1025° C., or from 900 to 1000° C. In some embodiments, the working point T4 is at least 800° C., at least 850° C., at least 875° C., or at least 900° C. In some embodiments, the working point T4 is at most 1100° C., for example at most 1050° C., at most 1025° C., or at most 1000° C.

In some embodiments, the glass transition temperature Tg is in a range from 525° C. to 725° C., for example from 550° C. to 700° C., from 575° C. to 675° C., or from 600° C. to 650° C. In some embodiments, the glass transition temperature Tg is at least 525° C., for example at least 550° C., at least 575° C., or at least 600° C. In some embodiments, the glass transition temperature Tg is at most 725° C., for example at most 700° C., at most 675° C., or at most 650° C.

When reference is made in the present disclosure to the mean coefficient of linear thermal expansion (or coefficient of thermal expansion, CTE), this means the mean coefficient of linear thermal expansion in a temperature range from 20° C. to 300° C., unless otherwise indicated.

In some embodiments, the mean coefficient of linear thermal expansion is in a range from 5.0 to 9.0 ppm/K, for example from 5.5 to 8.5 ppm/K, from 6.0 to 8.0 ppm/K, between 6.0 and 8.0 ppm/K, or from 6.5 to 7.5 ppm/K. In some embodiments, the mean coefficient of linear thermal expansion is at least 5.0 ppm/K, for example at least 5.5 ppm/K, at least 6.0 ppm/K, more than 6.0 ppm/K, or at least 6.5 ppm/K. In some embodiments, the mean coefficient of linear thermal expansion is at most 9.0 ppm/K, for example at most 8.5 ppm/K, at most 8.0 ppm/K, less than 8.0 ppm/K, or at most 7.5 ppm/K.

In some embodiments, the density of the glass is in a range from 4.00 to 4.60 g/cm³, for example from 4.05 to 4.55 g/cm³, from 4.10 to 4.50 g/cm³, from 4.15 to 4.45 g/cm³, from 4.20 to 4.40 g/cm³, or from 4.25 to 4.35 g/cm³. In some embodiments, the density of the glass is at least 4.00 g/cm³, for example at least 4.05 g/cm³, at least 4.10 g/cm³, at least 4.15 g/cm³, at least 4.20 g/cm³, or at least 4.25 g/cm³. In some embodiments, the density of the glass is at most 4.60 g/cm³, at most 4.55 g/cm³, at most 4.50 g/cm³, at most 4.45 g/cm³, at most 4.40 g/cm³, or at most 4.35 g/cm³.

Glass Article and/or Light Guide Element

The invention also relates to a glass article, in particular a light guide element, for example a glass fibre and/or a light guide rod, comprising or consisting of a glass of the invention. The light guide element can in particular be a glass fibre having a core glass and a cladding glass. The glass according to the invention is in particular used as core glass.

In some embodiments, the light guide element comprises at least one core glass and at least one cladding glass, for example precisely one core glass and at least one cladding glass, at least one core glass and precisely one cladding glass, or precisely one core glass and precisely one cladding glass. In some embodiments, the light guide element consists of at least one core glass and at least one cladding glass, for example of precisely one core glass and at least one cladding glass, of at least one core glass and precisely one cladding glass, or of precisely one core glass and precisely one cladding glass. In particular, the core glass comprises or consists of the glass of the present invention.

In some embodiments, the light guide element comprises the glass according to the invention as core glass, and also comprises a cladding glass surrounding the core glass.

In some embodiments, the light guide element is a light and/or image guide comprising the glass according to the invention as core glass, which is surrounded by a cladding glass. The light guide is for example a step-index fibre.

The light guide element can either be flexible or rigid. Whether it is a flexible or rigid light guide element is chiefly dependent on the diameter of the light guide element. The light guide element can also be present in the form of a glass fibre in a fibre bundle comprising a number of glass fibres. Such glass fibres and/or fibre bundles are usually flexible; the diameter of the individual glass fibres is usually a few dozen micrometres to a few hundred micrometres. Glass fibres generally consists of core-cladding systems having a core glass and a cladding glass surrounding the outer circumferential surface of the core glass. Light is guided by total internal reflection at the interface between core and cladding. In order to achieve total internal reflection, the cladding glass usually has a lower refractive index than the core glass.

Light guide rods are usually rigid since they usually have a larger diameter, from a little under 1 millimetre to a few centimetres. They can be configured as a core-cladding system as described above, but also as a glass rod without cladding glass, with total internal reflection taking place at the interface between the outer circumferential surface and the surrounding medium, i.e. generally air. A special form of light guide rod, which is also covered by the term and/or the invention, is the fibre rod, in which a number of glass elements composed of core-cladding systems are sintered together or fused together.

Glass fibres and/or fibre rods can transmit light and thus act as a light guide. However, they can also act as an image guide if there is a 1:1 correlation between the position of the individual fibre cores on the input surface and the position of the individual fibre cores on the output surface of the image guide. The terms are known to those skilled in the art and will not be explained in more detail below.

One relevant characteristic variable for forming a light guide element is the difference between the mean coefficient of linear thermal expansion of the core glass and the mean coefficient of linear thermal expansion of the cladding glass, also referred to as “ΔCTE(core-cladding)”. Depending on the application of the optical fibre, there are positive ΔCTE(core-cladding), but also a difference=0, and also negative differences. ΔCTE(core-cladding)≥0 ppm/K is preferred for light guides. ΔCTE(core-cladding)≥−0.5 ppm/K is preferred for image guides.

In some embodiments (in particular in light guides (LGF)), the core glass has a mean coefficient of linear thermal expansion which is greater than that of the cladding glass used. This makes it possible to improve the strength, in particular the fibre strength. In some embodiments, the difference between the mean coefficient of linear thermal expansion of the core glass and the mean coefficient of linear thermal expansion of the cladding glass is in a range from 0 to 5.5 ppm/K, for example from 0.1 to 5.0 ppm/K, from 0.2 to 4.5 ppm/K, from 0.5 to 4.0 ppm/K, from 1.0 to 3.5 ppm/K, from 1.2 to 3.0 ppm/K, from 1.5 to 2.6 ppm/K, or from >2.0 ppm/K to 2.4 ppm/K. In some embodiments, the difference between the mean coefficient of linear thermal expansion of the core glass and the mean coefficient of linear thermal expansion of the cladding glass is at least 0.1 ppm/K, for example at least 0.2 ppm/K, at least 0.5 ppm/K, at least 1.0 ppm/K, at least 1.2 ppm/K, at least 1.5 ppm/K, or more than 2.0 ppm/K. In some embodiments, the difference between the mean coefficient of linear thermal expansion of the core glass and the mean coefficient of linear thermal expansion of the cladding glass is at most 5.0 ppm/K, for example at most 4.5 ppm/K, at most 4.0 ppm/K, at most 3.5 ppm/K, at most 3.0 ppm/K, at most 2.6 ppm/K, or more than 2.4 ppm/K.

In some embodiments (in particular in image guides (LFB)), the difference between the mean coefficient of linear thermal expansion of the core glass and the mean coefficient of linear thermal expansion of the cladding glass is in a range from −0.5 to 3.5 ppm/K, for example from −0.4 to 3.0 ppm/K, from −0.3 to 2.5 ppm/K, from 0 to 2.0 ppm/K, or from 0.5 ppm/K to 1.5 ppm/K. In some embodiments, the difference between the mean coefficient of linear thermal expansion of the core glass and the mean coefficient of linear thermal expansion of the cladding glass is at least −0.5 ppm/K, for example at least −0.4 ppm/K, at least −0.3 ppm/K, at least −0.2 ppm/K, at least −0.1 ppm/K, at least 0 ppm/K, or at least 0.5 ppm/K. In some embodiments, the difference between the mean coefficient of linear thermal expansion of the core glass and the mean coefficient of linear thermal expansion of the cladding glass is at most 3.5 ppm/K, for example at most 3.0 ppm/K, at most 2.5 ppm/K, at most 2.0 ppm/K, or at most 1.5 ppm/K. In some embodiments, the cladding glass has a mean coefficient of linear thermal expansion in a range from 3.5 to 7.0 ppm/K, for example in a range from 4.0 to 6.5 ppm/K, or from 4.5 to 6.0 ppm/K. In some embodiments, the mean coefficient of linear thermal expansion of the cladding glass is at least 3.5 ppm/K, for example at least 4.0 ppm/K, or at least 4.5 ppm/K. In some embodiments, the mean coefficient of linear thermal expansion of the cladding glass is at most 7.0 ppm/K, for example at most 6.5 ppm/K, or at most 6.0 ppm/K.

The refractive index of the cladding glass is lower than that of the core glass, in order for the light to be reflected at the interface between core glass and cladding glass. In some embodiments, the difference between the refractive index n_{d} of the core glass and the refractive index n_{d} of the cladding glass is in a range from 0.05 to 0.40, for example from 0.08 to 0.35, from 0.10 to 0.30, from 0.15 to 0.27, or from 0.20 to 0.25. In some embodiments, the difference between the refractive index n_{d} of the core glass and the refractive index n_{d} of the cladding glass is at least 0.05, for example at least 0.08, at least 0.10, at least 0.15, or at least 0.20. In some embodiments, the difference between the refractive index n_{d} of the core glass and the refractive index n_{d} of the cladding glass is at most 0.40, for example at most 0.35, at most 0.30, at most 0.27, at most 0.26, or at most 0.25.

In some embodiments, the refractive index n_{d} of the cladding glass is in a range from 1.45 to 1.60, for example from 1.46 to 1.55, from 1.47 to 1.54, or from 1.48 to 1.52. In some embodiments, the refractive index n_{d} of the cladding glass is at least 1.45, for example at least 1.46, at least 1.47, or at least 1.48. In some embodiments, the refractive index n_{d} of the cladding glass is at most 1.60, for example at most 1.55, at most 1.54, or at most 1.52.

In some embodiments, the cladding glass has an SiO₂ content of >60 wt %, for example >65 wt % or at least 69 wt %. In some embodiments, the SiO₂ content is at most 75 wt %, for example up to 73 wt %. The cladding glass surrounds the glass according to the invention in the light and/or image guide. The glass according to the invention forms the “core glass”. The cladding glass therefore tends to be exposed to stronger environmental influences than the core glass. A higher SiO₂ content affords better chemical resistance. Therefore, the content of this component is preferably higher in the cladding glass than in the core glass.

In some embodiments, the cladding glass also comprises at least 5.5 wt % of alkali metal oxides. In some embodiments, the content of alkali metal oxides in the cladding glass is at least 7 wt %, for example at least 8 wt %. Alkali metal oxides include in particular Na₂O, K₂O, Li₂O.

In some embodiments, the Na₂O content is at least 0.5 wt %, for example at least 2 wt %. In some embodiments of the cladding glass, the cladding glass contains at least 6 wt % Na₂O. In some embodiments, the Na₂O content is at most 15.5 wt %, for example at most 15 wt %.

In some embodiments, Li₂O is present in the cladding glass at a proportion of up to 0.7 wt %, for example up to 0.6 wt %. In some embodiments, the cladding glass is free from Li₂O.

In some embodiments, the K₂O content in the cladding glass is at least 2 wt %, for example at least 2.5 wt %. In some embodiments, the K₂O content is at most 8 wt %, for example up to 7.5 wt %. In some embodiments, the glass is free from K₂O. In some embodiments, the cladding glass no alkali metal oxides other than Na₂O and K₂O.

In some embodiments, the cladding glass comprises at least 0.5 wt % of oxides selected from the group consisting of CaO, MgO, BaO and ZnO and mixtures thereof. In some embodiments, the total content of these oxides is at least 0.6 wt %. In some embodiments, the total content of such oxides is at most 12 wt %, for example at most 11 wt %, at most 5 wt % or at most 2.5 wt %. In some embodiments, the cladding glass comprises precisely two oxides selected from CaO, MgO, BaO and ZnO. In some embodiments, the cladding glass comprises only one oxide selected from the group consisting of CaO, MgO, BaO and ZnO.

In some embodiments, the cladding glass comprises Al₂O₃ at a content of at least 0.5 wt %, for example at least 1 wt %, or at least 2 wt %. In some embodiments, the cladding glass comprises at most 7.5 wt %, for example up to 7 wt %, at most 3 wt %, or at most 1 wt %, Al₂O₃.

The cladding glass can comprise B₂O₃, wherein, in some embodiments, at least 9 wt % or at least 9.5 wt % B₂O₃ are contained in the cladding glass. In some embodiments, the cladding glass contains at most 19 wt %, for example up to 18.5 wt %, B₂O₃.

In some embodiments, the cladding glass comprises a higher content of the sum of the components B₂O₃ and Al₂O₃ than the core glass.

In some embodiments, the SiO₂ content in the cladding glass is higher than the SiO₂ content in the core glass. Furthermore, in some embodiments, the La₂O₃ content in the cladding glass is much lower than in the core glass. Excessively high proportions of SiO₂ in the core mean it is no longer possible to adjust the relatively high refractive index if said refractive index is adjusted using La₂O₃. Furthermore, in some embodiments, the ZnO content is the cladding glass is much lower than in the core glass. This is because the viscosity of the core glass is preferably lower than that of the cladding glass. This improves fibre drawing properties. In some embodiments, the sum of the contents of ZnO and BaO in the cladding glass is lower than this sum in the core glass.

The cladding glass can contain ZrO₂, wherein, in some embodiments, at most 0.04 wt % or up to 0.03 wt % ZrO₂ is present in the cladding glass.

As₂O₃ can for example be contained in the cladding glass at a content of up to 0.05 wt %, or up to 0.01 wt %. Arsenic oxide is responsible for solarization. In some embodiments, the cladding glass is free from As₂O₃.

Sb₂O₃ can for example be contained in the cladding glass at a content of up to 0.05 wt %, or up to 0.01 wt %. In some embodiments, the cladding glass is free from Sb₂O₃.

The cladding glass can also comprise fluorine or fluoride, and/or chlorine or chloride. In some embodiments, the fluoride content is up to 0.6 wt %, or up to 0.55 wt %. Chloride can be contained in the cladding glass at a content, for example, of at most 0.2 or up to 0.15 wt %. Some embodiments of the cladding glass are free from fluorine or fluoride and/or chlorine or chloride.

The following table shows some preferred embodiments of cladding glasses which can be used together with the glasses according to the invention. The cladding glasses contain (in wt %):

| Component | Group 1 | Group 2 | Group 3 | Group 4 |
|---|---|---|---|---|
| SiO₂ | 70-78 | 63-75 | 75-85 | 62-70 |
| Al₂O₃ | 5-10 | 1-7 | 1-5 | 1-10 |
| B₂O₃ | 5-14 | 0-3 | 10-14 | >15 |
| Li₂O | free | 0-1 | 0-3 | <0.1 |
| Na₂O | 0-10 | 8-20 | 2-8 | 0-10 |
| K₂O | 0-10 | 0-6 | 0-1 | 0-10 |
| MgO | 0-1 | 0-5 | free | 0-5 |
| CaO | 0-2 | 1-9 | free | 0-5 |
| SrO | 0-1 | free | free | 0-5 |
| BaO | 0-1 | 0-5 | free | 0-5 |
| F | 0-1 | 0-1 | free | 0-1 |

Those skilled in the art are able to use yet other cladding glasses on the basis of their knowledge in the art.

A borosilicate glass which has proven particularly advantageous as cladding glass is one which, with the above-described variants for the core glass, results in a robust wide-angle fibre, i.e. one with good tensile strength and an advantageous numerical aperture NA of 0.86, corresponding to a 2α angular aperture of 120°.

In some embodiments, the numerical aperture NA of the fibre is in a range from 0.40 to 1.30, for example from 0.45 to 1.20, from 0.50 to 1.10, from 0.60 to 1.05, from 0.70 to 1.00, from 0.75 to 0.95, from 0.77 to 0.93, or from 0.80 to 0.90. In some embodiments, the numerical aperture of the fibre is at least 0.40, for example at least 0.45, at least 0.50, 0.60, at least 0.70, at least 0.75, at least 0.77, or at least 0.80. In some embodiments, the numerical aperture of the fibre is at most 1.30, for example at most 1.20, at most 1.10, at most 1.05, at most 1.00, at most 0.95, for example at most 0.93, or at most 0.90.

In some embodiments, the 2α angular aperture of the fibre is in a range from 95° to 140°, for example from 100° to 135°, from 105° to 130°, from 110° to 125°, or from 115° to 120°. In some embodiments, the angular aperture of the fibre is at least 95°, for example at least 100°, at least 105°, at least 110°, or at least 115°. In some embodiments, the angular aperture of the fibre is at most 140°, for example at most 135°, at most 130°, at most 125°, or at most 120°.

In particular for endoscopic applications with camera chips, the diagonal viewing angle of which corresponds to approximately 120°, a 2α angular aperture of 100°, preferably of 120°, which corresponds to an NA of 0.86, is particularly advantageous.

In some embodiments, the fibre has a length in a range from 0.1 to 50 m, for example from 0.2 to 25 m, from 0.5 to 10 m, from 1.0 to 5.0 m, from 1.0 to 3.0 m, or from 1.5 to 2.5 m. In some embodiments, the fibre has a length of at least 0.1 m, for example at least 0.2 m, at least 0.5 m, at least 1.0 m, or at least 1.5 m. In some embodiments, the fibre has a length of at most 50 m, for example at most 25 m, at most 10 m, at most 5.0 m, at most 3.0 m, or at most 2.5 m. Of course, such fibres can also be provided in considerably longer lengths in a fibre drawing method. For example, it is entirely customary to first produce lengths of greater than 50 m; a few 100s of metres to a few kilometres, and then to tailor these for example to the lengths mentioned above.

In some embodiments, the fibre has a diameter in a range from 2.0 to 1000 μm, for example in a range from 3.0 to 750 μm, from 4.0 to 500 μm, from 10 to 425 μm, from 20 to 350 μm, from 25 to 150 μm, or from 35 to 100 μm. In some embodiments, the fibre has a diameter of at least 2.0 μm, for example at least 3.0 μm, at least 4.0 μm, at least 10 μm, at least 20 μm, at least 25 μm, or at least 35 μm. In some embodiments, the fibre has a diameter of at most 1000 μm, for example at most 750 μm, at most 500 μm, at most 425 μm, at most 350 μm, at most 150 μm, or at most 100 μm.

In some embodiments (in particular in light guides (LGF)), the fibre has a diameter in a range from 4.0 to 1000 μm, for example from 10 to 350 μm, from 15 to 150 μm, from 20 to 100 μm, or from 25 to 75 μm. In some embodiments (in particular in light guides (LGF)), the fibre has a diameter of at least 4.0 μm, for example at least at least 10 μm, at least 15 μm, at least 20 μm, or at least 25 μm. In some embodiments (in particular in light guides (LGF)), the fibre has a diameter of at most 1000 μm, for example at most 350 μm, at most 150 μm, at most 100 μm, or at most 75 μm.

In some embodiments (in particular in image guides (LFB)), the fibre has a diameter in a range from 2.0 to 10 μm, for example from 3.0 to 7.0 μm, or from 4.0 to 6.0 μm. In some embodiments (in particular in image guides (LFB)), the fibre has a diameter of at least 2.0 μm, for example at least 3.0 μm, or at least 4.0 μm. In some embodiments (in particular in image guides (LFB)), the fibre has a diameter of at most 10 μm, for example at most 7.0 μm, or at most 6.0 μm.

The invention also relates to a fibre bundle comprising one or more fibres according to the invention, or to a fibre bundle consisting of two or more fibres according to the invention.

In some embodiments, such fibre bundles have a length in a range from 0.1 to 50 m, for example from 0.2 to 25 m, from 0.5 to 10 m, from 1.0 to 5.0 m, from 1.0 to 3.0 m, or from 1.5 to 2.5 m. In some embodiments, the fibre bundles have a length of at least 0.1 m, for example at least 0.2 m, at least 0.5 m, at least 1.0 m, or at least 1.5 m. In some embodiments, the fibre bundles have a length of at most 50 m, for example at most 25 m, at most 10 m, at most 5.0 m, at most 3.0 m, or at most 2.5 m.

In some embodiments (in particular in image guides, for example LFB), the fibre bundle has 100 to 50 000 fibres, for example 200 to 40 000, 500 to 25 000, 1000 to 15 000, or 3000 to 10 000 fibres. In some embodiments (in particular in image guides, for example LFB), the fibre bundle has at least 100 fibres, for example at least 200, at least 500, at least 1000, at least 3000, at least 5000, at least 7500, or at least 10 000 fibres. In some embodiments (in particular in image guides, for example LFB), the fibre bundle has at most 100 000 fibres, for example at most 50 000, at most 40 000, at most 25 000, at most 15 000, or at most 10 000 fibres.

In some embodiments (in particular in light guides (LGF)), the fibre bundle has 3 to 1000 fibres, for example 5 to 750, 10 to 500, 20 to 200, or 50 to 100 fibres. In some embodiments (in particular in light guides (LGF)), the fibre bundle has at least 3 fibres, for example at least at least 5, at least 10, at least 20, or at least 50 fibres. In some embodiments (in particular in light guides (LGF)), the fibre bundle has at most 1000 fibres, for example at most 750, at most 500, at most 200, or at most 100 fibres.

### Production Method

The invention also relates to a method for producing a glass or light guide element according to the invention. In particular, the method comprises the following steps:
melting the glass raw materials,
cooling the glass obtained, a glass or light guide element of the invention being obtained.

In some embodiments, the method comprises the step of refining the glass melt. In some embodiments, the refining temperature is in a range from 1150° C. to 1650° C., for example from 1200° C. to 1600° C., from 1225° C. to 1550° C., from 1250° C. to 1500° C., from 1275° C. to 1450° C., from 1300° C. to 1400° C., or from 1320° C. to 1360° C. In some embodiments, the refining temperature is at least 1150° C., for example at least 1200° C., at least 1225° C., at least 1250° C., at least 1275° C., at least 1300° C., or at least 1320° C. In some embodiments, the refining temperature is at most 1650° C., for example at most 1600° C., at most 1550° C., at most 1500° C., at most 1450° C., at most 1400° C., or at most 1360° C.

The glass batch can be melted and/or refined using high-frequency (HF) heating. However, it is also possible to melt and refine the batch without high-frequency heating. It is a particular advantage of the glass of the present invention that expensive high-frequency heating can be dispensed with during the production thereof. Dispensing with high-frequency heating is also advantageous since, in the presence of increasing contents of Nb and Ti, HF-refined glass becomes brown or yellow/green. If the glass contains Fe as impurity, HF-refining leads to an increased content of Fe(II) which absorbs in the near infrared region (NIR).

“High-frequency heating” means a method in which the batch to be heated is heated by inductively coupling-in an alternating electromagnetic field. The alternating electromagnetic field has for example frequencies of at least 50 kHz or at least 100 kHz. The frequency is for example at most 5 MHz, at most 3 MHz, or at most 1 MHz. The electromagnetic field generates alternating currents in the electrically conductive glass melt, which lead to direct heating of the melt due to resistive heating.

The cooling step can comprise active cooling, passive cooling, or a mixture of both.

In some embodiments, the method comprises the step of processing the glass melt, in particular by means of down draw, overflow fusion, floating or tube drawing, in particular the Danner method, Vello method or vertical drawing method.

In some embodiments, the method comprises the step of manufacturing a light guide element using a fibre drawing method or redrawing, to give a glass fibre or light guide fibre, to give a lens preform or to give a container. In some embodiments, the method comprises the step of manufacturing a glass article using a fibre drawing method to give a glass fibre.

### Use

The invention also relates to the use of a glass or glass article according to the invention as fibre glass. The invention also relates to the use of a glass or light guide element according to the invention in or as fibre glass. The invention also relates to the use of a glass according to the invention as core glass, in particular as core glass in a light and/or image guide. The invention also relates to the use of a glass or glass article according to the invention as or in a light and/or image guide.

The invention also relates to the use of a glass or glass article according to the invention in the fields of imaging, projection, telecommunications, optical data transmission technology, mobile drive, laser technology and disinfection, and also optical elements or preforms of such optical elements.

The use in endoscopic systems for industrial and/or medical technology, in particular for single-use endoscopes in the medical field, is particularly advantageous.

### Exemplary Configurations

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, and wherein, for a sample thickness of 25 mm and a wavelength of 380 nm, the glass has a pure transmittance of at least 0.900.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, and wherein the lower devitrification point LDP is at least 650° C. when the glass is thermally treated for a retention time of 5 minutes in a gradient furnace with rising temperature control.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂₃, wherein the ratio of the sum of the proportions by weight of Gd₂O₃ and Y₂O₃ to the proportion by weight of SiO₂ is at least 0.01, and wherein the difference between the lower devitrification point LDP and the softening point T7.6 is at least 50 K.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, and wherein the refractive index n_{d} of the glass is in a range from 1.65 to 1.80.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, wherein, for a sample thickness of 25 mm and a wavelength of 380 nm, the glass has a pure transmittance of at least 0.900, and wherein the lower devitrification point LDP is at least 650° C. when the glass is thermally treated for a retention time of 5 minutes in a gradient furnace with rising temperature control.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, wherein, for a sample thickness of 25 mm and a wavelength of 380 nm, the glass has a pure transmittance of at least 0.900, and wherein the difference between the lower devitrification point LDP and the softening point T7.6 is at least 50 K.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, wherein, for a sample thickness of 25 mm and a wavelength of 380 nm, the glass has a pure transmittance of at least 0.900, and wherein the refractive index n_{d} of the glass is in a range from 1.65 to 1.80.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, and wherein the lower devitrification point LDP is at least 650° C. when the glass is thermally treated for a retention time of 5 minutes in a gradient furnace with rising temperature control, and wherein the difference between the lower devitrification point LDP and the softening point T7.6 is at least 50 K.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, wherein the lower devitrification point LDP is at least 650° C. when the glass is thermally treated for a retention time of 5 minutes in a gradient furnace with rising temperature control, and wherein the refractive index n_{d} of the glass is in a range from 1.65 to 1.80.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, wherein the difference between the lower devitrification point LDP and the softening point T7.6 is at least 50 K and wherein the refractive index n_{d} of the glass is in a range from 1.65 to 1.80.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, wherein, for a sample thickness of 25 mm and a wavelength of 380 nm, the glass has a pure transmittance of at least 0.900, and wherein the lower devitrification point LDP is at least 650° C. when the glass is thermally treated for a retention time of 5 minutes in a gradient furnace with rising temperature control, and wherein the difference between the lower devitrification point LDP and the softening point T7.6 is at least 50 K.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, wherein, for a sample thickness of 25 mm and a wavelength of 380 nm, the glass has a pure transmittance of at least 0.900, wherein the lower devitrification point LDP is at least 650° C. when the glass is thermally treated for a retention time of 5 minutes in a gradient furnace with rising temperature control, and wherein the refractive index n_{d} of the glass is in a range from 1.65 to 1.80.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, wherein, for a sample thickness of 25 mm and a wavelength of 380 nm, the glass has a pure transmittance of at least 0.900, wherein the difference between the lower devitrification point LDP and the softening point T7.6 is at least 50 K, and wherein the refractive index n_{d} of the glass is in a range from 1.65 to 1.80.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, wherein the lower devitrification point LDP is at least 650° C. when the glass is thermally treated for a retention time of 5 minutes in a gradient furnace with rising temperature control, wherein the difference between the lower devitrification point LDP and the softening point T7.6 is at least 50 K, and wherein the refractive index n_{d} of the glass is in a range from 1.65 to 1.80.

Some preferred embodiments relate to a glass or a glass article comprising the following components in the stated proportions (by weight):

| Component | Min (wt %) | Max (wt %) |
|---|---|---|
| SiO₂ | 18 | 35 |
| B₂O₃ | 1.0 | 13 |
| CaO | 0 | 0.5 |
| BaO | 2.0 | 30 |
| SrO | 0 | 2.0 |
| ZnO | 1.0 | 18 |
| La₂O₃ | 5.0 | 40 |
| Gd₂O₃ | 0 | 8.0 |
| Y₂O₃ | 0 | 8.0 |
| ZrO₂ | 1.0 | 7.0 |
| Ta₂O₅ | 0 | 7.0 |
| Nb₂O₅ | 0 | 7.0 |
| Σ R₂O | 0 | 2.0 |

wherein the glass comprises at least one of the two components Gd₂O₃ and Y₂O₃, wherein the ratio of the sum of the proportions by weight of Gd₂O₃ and Y₂O₃ to the proportion by weight of SiO₂ is at least 0.01, wherein, for a sample thickness of 25 mm and a wavelength of 380 nm, the glass has a pure transmittance of at least 0.900, wherein the lower devitrification point LDP is at least 650° C. when the glass is thermally treated for a retention time of 5 minutes in a gradient furnace with rising temperature control, wherein the difference between the lower devitrification point LDP and the softening point T7.6 is at least 50 K, and wherein the refractive index n_{d} of the glass is in a range from 1.65 to 1.80.

As previously described, it is particularly advantageous if both Gd₂O₃ and Y₂O₃ are present in the glass according to the invention.

### EXAMPLES

### 1. Exemplary Glasses and Properties Thereof

The following table shows synthesis compositions and properties of selected glasses. The proportions of the individual components relate to the proportions in wt % in the synthesis composition. They are each normalized to 100% and, except for the refining agent, rounded to one decimal place. Deviations from 100% are due to rounding. The pure transmittance Ti at 380 nm and at 600 nm is given in each case for a sample thickness of 25 mm and rounded to three decimal places. The examples were produced under laboratory conditions, which are associated with decreased internal quality. Even higher pure transmittance can be achieved with the glass compositions of the invention under production conditions than was achieved in the present examples.

| | | | | |
|---|---|---|---|---|
| Comp. Ex. A | Comp. Ex. B | Ex. 1 | Ex. 2 | Ex. 3 |

Component
(wt %)

| | | | | | |
|---|---|---|---|---|---|
| SiO₂ | 29.5 | 29.5 | 30.3 | 30.2 | 28.1 |
| B₂O₃ | 3.8 | 3.8 | 3.1 | 4.0 | 3.4 |
| Li₂O | 0.2 | 0.2 | 1.0 | 1.0 | 0.8 |
| Na₂O | 0.6 | 0.6 | 1.0 | | |
| BaO | 21.4 | 21.5 | 14.4 | 21.6 | 21.2 |
| ZnO | 13.8 | 13.9 | 1.9 | 2.1 | 13.5 |
| La₂O₃ | 21.8 | 21.8 | 24.8 | 26.9 | 20.7 |
| Gd₂O₃ | | | 5.2 | | 2.9 |
| Y₂O₃ | | | 6.8 | 1.0 | 1.0 |
| ZrO₂ | 2.4 | 2.4 | 2.6 | 5.7 | 3.4 |
| Ta₂O₅ | | | 2.6 | 2.4 | 4.8 |
| Nb₂O₅ | 6.2 | 6.2 | 6.2 | 5.1 | 0.4 |
| Sb₂O₃ | 0.30 | 0.05 | 0.05 | 0.05 | 0.05 |

Properties

| | | | | | |
|---|---|---|---|---|---|
| Ti (380 nm) | 0.749 | 0.895 | 0.857 | 0.866 | 0.889 |
| Ti (600 nm) | 0.995 | 0.997 | 0.997 | 0.998 | 1.000 |
| n_{d} | 1.718 | 1.717 | 1.730 | 1.726 | 1.731 |
| v_{d} | 45.5 | 45.6 | 45.7 | | 47.4 |
| Density [g/cm³] | 4.12 | | 4.15 | 4.13 | 4.20 |
| CTE [ppm/K] | 7.2 | | 7.6 | 7.4 | 7.5 |
| Tg [° C.] | 641 | | 665 | 668 | 635 |

As shown in the table, the examples and comparative examples largely correspond in terms of refractive index n_{d}, Abbe number v_{d}, density, mean coefficient of linear thermal expansion (CTE) in the temperature range from 20° C. to 300° C. and glass transition temperature Tg. CTE, density and Tg were tested on samples which had been cooled at a rate of 120 K/min before the measurement. Refractive index n_{d} and Abbe number v_{d} were tested on samples which had been cooled at a rate of 30 K/min before the measurement. If such samples are cooled again before the measurement, the cooling thereof generally starts from a temperature above the Tg (approx. 100 K) but below the softening point of the glass.

The following table show synthesis compositions and properties of further exemplary glasses.

| | | | | | |
|---|---|---|---|---|---|
| Ex. 4 | Ex. 5 | Ex. 6 | Ex. 7 | Ex. 8 | Ex. 9 |

Component (wt %)

| | | | | | | |
|---|---|---|---|---|---|---|
| SiO₂ | 27.0 | 28.7 | 20.1 | 29.2 | 27.9 | 28.4 |
| B₂O₃ | 5.0 | 3.4 | 13.0 | 3.5 | 3.4 | 3.4 |
| Li₂O | | 0.3 | 1.1 | 0.3 | 0.8 | |
| Na₂O | | | 0.5 | | | |
| BaO | 26.4 | 21.2 | 11.6 | 21.6 | 21.2 | 20.2 |
| SrO | 2.0 | | | | | |
| ZnO | 1.1 | 14.6 | 7.9 | 12.7 | 13.9 | 14.4 |
| La₂O₃ | 34.2 | 20.2 | 29.5 | 20.6 | 19.7 | 20.2 |
| Gd₂O₃ | 0.5 | 1.9 | 5.7 | | 3.8 | 2.9 |
| Y₂O₃ | 0.6 | 1.4 | 6.3 | 1.0 | 1.0 | 1.0 |
| ZrO₂ | 1.9 | 2.9 | 2.1 | 3.4 | 3.4 | 2.9 |
| Ta₂O₅ | 0.3 | 4.8 | | 6.9 | 4.8 | 6.7 |
| Nb₂O₅ | 1.0 | 0.6 | 2.1 | 0.8 | 0.2 | |
| Sb₂O₃ | 0.05 | 0.05 | 0.05 | 0.05 | 0.05 | 0.05 |

Properties

| | | | | | | |
|---|---|---|---|---|---|---|
| Ti (380 nm) | 0.921 | 0.927 | 0.939 | 0.941 | 0.944 | 0.946 |
| Ti (600 nm) | 0.997 | 1.000 | 0.997 | 0.997 | 1.000 | 0.994 |
| n_{d} | 1.720 | | 1.730 | 1.714 | 1.721 | 1.717 |
| v_{d} | 50.0 | | 49.3 | 47.4 | 47.8 | 47.6 |
| Density | 4.34 | 4.20 | 4.20 | 4.24 | 4.24 | 4.33 |
| [g/cm³] | | | | | | |
| CTE [ppm/K] | 8.5 | 7.0 | 7.8 | 6.9 | 7.2 | 6.8 |
| Tg [° C.] | 707 | 655 | 597 | 667 | 638 | 693 |

The following table show synthesis compositions and properties of further exemplary glasses.

| | | | | | |
|---|---|---|---|---|---|
| Ex. 10 | Ex. 11 | Ex. 12 | Ex. 13 | Ex. 14 | Ex. 15 |

Component (wt %)

| | | | | | | |
|---|---|---|---|---|---|---|
| SiO₂ | 28.7 | 28.7 | 28.0 | 28.6 | 28.7 | 28.0 |
| B₂O₃ | 3.4 | 3.5 | 3.4 | 3.4 | 3.4 | 3.4 |
| Li₂O | 0.3 | | 0.8 | 0.3 | 0.3 | 0.8 |
| BaO | 21.2 | 20.5 | 21.2 | 21.2 | 21.2 | 21.2 |
| ZnO | 13.5 | 13.6 | 13.9 | 13.5 | 13.5 | 13.9 |
| La₂O₃ | 19.2 | 20.5 | 19.7 | 19.3 | 20.2 | 19.7 |
| Gd₂O₃ | 2.9 | 3.9 | 3.8 | 2.9 | 2.9 | 3.8 |
| Y₂O₃ | 1.0 | | 1.0 | 1.0 | 1.4 | 1.0 |
| ZrO₂ | 2.9 | 2.9 | 3.4 | 2.9 | 3.2 | 3.4 |
| Ta₂O₅ | 6.3 | 6.3 | 4.7 | 6.3 | 4.8 | 4.7 |
| Nb₂O₅ | 0.8 | | 0.2 | 0.8 | 0.4 | 0.2 |
| Sb₂O₃ | 0.05 | 0.05 | 0.05 | 0.05 | 0.05 | 0.05 |

Properties

| | | | | | | |
|---|---|---|---|---|---|---|
| Ti (380 nm) | 0.946 | 0.947 | 0.953 | 0.966 | 0.969 | 0.971 |
| Ti (600 nm) | 0.997 | 0.991 | 0.998 | 0.998 | 0.999 | 1.000 |
| n_{d} | 1.717 | 1.713 | 1.719 | 1.718 | 1.717 | 1.719 |
| v_{d} | 47.3 | 47.9 | 47.9 | 47.3 | 47.9 | 47.9 |
| Density | 4.30 | 4.30 | 4.32 | 4.31 | 4.30 | |
| [g/cm³] | | | | | | |
| CTE [ppm/K] | 6.9 | 6.8 | 7.3 | 7.0 | 7.0 | |
| Tg [° C.] | 663 | 690 | 635 | 659 | 662 | |

Melting conditions can also lead, for the same composition, to slightly differing transmittance values. A decrease in transmittance can for example be observed when melting in or with Pt. This applies particularly to small-volume melts, for example laboratory scale melts.

The following table shows the analytical composition of selected glasses (in wt %). The compositions were analyzed by means of X-ray fluorescence spectroscopy (XRF).

| | Comp. | Comp. | | | | |
|---|---|---|---|---|---|---|
| Component (wt %) | Ex. A | Ex. B | Ex. 12 | Ex. 13 | Ex. 14 | Ex. 15 |
| SiO₂ | 29.8 | 29.9 | 28.8 | 29.6 | 29.6 | 28.8 |
| B₂O₃ | 3.9 | 3.6 | 3.2 | 3.4 | 3.2 | 3.2 |
| Li₂O | 0.2 | 0.2 | 0.7 | 0.3 | 0.3 | 0.7 |
| Na₂O | 0.7 | 0.7 | | | | |
| BaO | 21.9 | 21.8 | 21.4 | 21.3 | 21.4 | 21.5 |
| SrO | | | 0.02 | 0.2 | 0.02 | 0.02 |
| ZnO | 13.7 | 13.9 | 13.5 | 13.0 | 13.0 | 13.4 |
| La₂O₃ | 21.3 | 21.7 | 19.7 | 19.2 | 20.2 | 19.7 |
| Gd₂O₃ | | | 3.8 | 2.8 | 2.9 | 3.8 |
| Y₂O₃ | | | 0.9 | 0.9 | 1.4 | 0.9 |
| ZrO₂ | 2.3 | 2.4 | 3.2 | 2.7 | 3.0 | 3.2 |
| Ta₂O₅ | | | 4.5 | 6.0 | 4.6 | 4.5 |
| Nb₂O₅ | 6.0 | 6.1 | 0.2 | 0.7 | 0.4 | 0.2 |
| Sb₂O₃ | 0.27 | 0.04 | 0.04 | 0.04 | 0.04 | 0.04 |

The following table shows further properties of examples 12 to 14.

| Property | Ex. 12 | Ex. 13 | Ex. 14 |
|---|---|---|---|
| Annealing point T13 [° C.] | 643 | 669 | 671 |
| Softening point T7.6 [° C.] | 776 | 804 | 806 |
| Working point T4 [° C.] | 952 | 979 | 982 |

The annealing point T13 is the temperature at which the viscosity is 10¹³ dPas. The softening point T7.6 is the temperature at which the viscosity is 10^{7.6} dPas. The working point T4 is the temperature at which the viscosity is 10⁴ dPas.

### 2. Resistance to Crystallization

The resistance to crystallization of exemplary glasses 12 to 14 was tested.

The rate of crystallization was determined by thermally treating the glass for a retention time of 5 minutes or 60 minutes in a gradient furnace with rising temperature control. The rate of crystallization was determined using glass grain having a diameter of 1.6 mm to 4 mm. For the thermal treatment in the gradient furnace, glass grain was placed on a platinum support. The support had a depression for receiving a grain of glass. A hole on the underside of each depression made it possible to microscopically determine the rate of crystallization following the thermal treatment. The highest rate of crystallization which was ascertained is the maximum rate of crystallization KGₘₐₓ. LDP and UDP were determined as the lower and upper limits, respectively, of the temperature range in which crystallization took place.

The results are summarized in the following tables.

| | | | |
|---|---|---|---|
| Property | Ex. 12 | Ex. 13 | Ex. 14 |

5 minutes' retention time

| | | | |
|---|---|---|---|
| LDP [° C.] | <895 | <885 | <895 |
| UDP [° C.] | 1075 | 1090 | 1115 |
| KGₘₐₓ [μm/min] | 9.6 | 3.9 | 11.4 |
| T (KGₘₐₓ) [° C.] | 1035 | 1050 | 1030 |

60 minutes' retention time

| | | | |
|---|---|---|---|
| LDP [° C.] | n.d. | n.d. | n.d. |
| UDP [° C.] | 1160 | 1130 | 1155 |
| KGₘₐₓ [μm/min] | 2.4 | 3.1 | 1.8 |
| T (KGₘₐₓ) [° C.] | 985 | 1035 | 980 |

LDP could not be determined (n.d.) for 60 minutes' retention time.

### 3. Cladding Glass and Core Glass

Borosilicate glasses are particularly suitable for cladding glass for the combination of glasses described herein. Borosilicate glasses are generally glasses containing SiO₂ and B₂O₃. In particular, borosilicate glasses contain SiO₂ from 60 to 75%, B₂O₃ from 7 to 25%, and Al₂O₃ from 5 to 17%. Further components, in particular such as alkalis, can also be present.

Modified borosilicate glasses are also possible, which for example may have lower contents of B₂O₃. The composition ranges B1 and B2 listed in the table below are advantageous for combination with the referring core glasses described herein, in particular as cladding glass (figures given in wt %):

| Composition | B1 | B2 |
|---|---|---|
| SiO₂ | 60-75 | 60-75 |
| B₂O₃ | 7-25 | 0.5-15 |
| Na₂O | 0-8 | 1-15 |
| K₂O | 0-8 | 0-15 |
| Al₂O₃ | 5-17 | 3-10 |

Of course, further optional components, in particular such as MgO and/or TiO₂ and/or CaO are possible.

The following table lists two cladding glasses with the composition thereof (according to analysis, in wt % based on oxide) as further exemplary embodiments. These generally belong to group B2 of the aforementioned table of cladding glass types. Furthermore, n_{d} means refractive index, CTE means the mean coefficient of linear thermal expansion in the range from 20° C. to 300° C., softening point T7.6 means the temperature at a viscosity of 10^{7,6} dPas, A is acid resistance (loss of weight after acid attack in order to classify the glasses into acid classes), B is base resistance (resistance of glasses to boiling aqueous mixed alkali solution).

Example

| | I | II |
|---|---|---|
| Cladding glass type | Group 1 | Group 2 |
| SiO₂ | 73.9 | 69.9 |
| B₂O₃ | 9.60 | 1.0 |
| Na₂O | 6.60 | 12.6 |
| K₂O | 2.56 | 3.2 |
| MgO | 0.01 | 2.7 |
| CaO | 0.63 | 5.1 |
| BaO | 0.04 | 2.1 |
| Al₂O₃ | 6.62 | 4.0 |
| TiO₂ | | 0.1 |
| F | 0.08 | 0.2 |
| Cl | 0.18 | |
| Fe₂O₃ | 0.04 | |
| Sb₂O₃ | <0.005 | |
| As₂O₃ | <0.005 | 0.1 |
| Sum | 100.26 | 101 |
| Properties | | |
| n_{d} | 1.49 | 1.514 |
| CTE [ppm/K] | 5.5 | 9.1 |
| T7.6 [° C.] | 790 | 720 |
| A [class] | 1 | 1 |
| B [class] | 2 | 2 |

The numerical aperture (NA) and angular aperture of a fibre optic light guide (light guide fibre) can be calculated based on the difference in the refractive index between core glass and cladding glass. The results are summarized in the following table for combinations of glasses of the invention as core glass (see above, examples 1 to 4 and 6 to 15) with the cladding glasses I and II. The expression “Δ CTE” denotes the difference between CTE of the core glass and CTE of the cladding glass.

| | Core glass | | | | |
|---|---|---|---|---|---|
| | Cladding glass I | | | Cladding glass II | |
| | | Angular | Δ CTE | | Angular |
| | NA | aperture [°] | [ppm/K] | NA | aperture [°] |
| Ex. 1 | 0.88 | 123 | 2.1 | 0.84 | 114 |
| Ex. 2 | 0.87 | 121 | 1.9 | 0.83 | 112 |
| Ex. 3 | 0.88 | 124 | 2.0 | 0.84 | 114 |
| Ex. 4 | 0.86 | 119 | 3.0 | 0.82 | 109 |
| Ex. 6 | 0.88 | 123 | 2.3 | 0.84 | 114 |
| Ex. 7 | 0.85 | 116 | 1.4 | 0.80 | 107 |
| Ex. 8 | 0.86 | 119 | 1.7 | 0.82 | 110 |
| Ex. 9 | 0.85 | 117 | 1.3 | 0.81 | 108 |
| Ex. 10 | 0.85 | 117 | 1.4 | 0.81 | 108 |
| Ex. 11 | 0.85 | 115 | 1.3 | 0.80 | 107 |
| Ex. 12 | 0.86 | 118 | 1.8 | 0.81 | 109 |
| Ex. 13 | 0.85 | 117 | 1.5 | 0.81 | 108 |
| Ex. 14 | 0.85 | 117 | 1.5 | 0.81 | 108 |
| Ex. 15 | 0.86 | 118 | | 0.81 | 109 |

### BRIEF DESCRIPTION OF THE DRAWINGS

The invention is intended to be explained below on the basis of the figures. The figures are also exemplary embodiments,

and show

FIG. 1: a schematic depiction of a glass fibre,

FIG. 2: in a first line chart, the spectral attenuation of the glass fibre according to the invention compared to a conventional glass fibre, and

FIG. 3: in a second line chart, the measured intensity relative to the angle to the radiation axis of the glass fibre, for determining the angular aperture.

### DETAILED DESCRIPTION

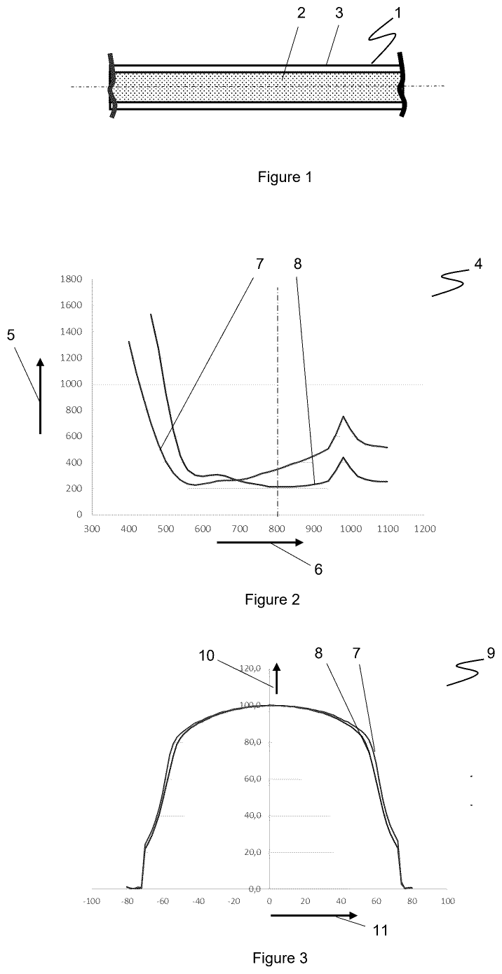

FIG. 1 schematically depicts a glass fibre in the form of a light guide element 1, the glass fibre having a core glass 2 and a cladding glass 3. The total diameter of this fibre produced in this way is 70 μm. In the exemplary embodiment show, the glass from the above example 15 was used as core glass 2.

The cladding glass 3 consists of a borosilicate glass which was described above, in particular with compositions containing the components of groups B1 or B2, and is typically in the form of a glass tube.

FIG. 2 shows, in a first line chart 4, the results of spectral attenuation measurements for a conventional glass fibre 7 and the glass fibre 8 according to the invention, as described previously with respect to FIG. 1. The spectral attenuation 5 is plotted here relative to the wavelength 6 (in nm) of the transmitted light. To this end, what are referred to as measurement light guides of specific lengths, e.g. 1 m or 3 m long, were produced, and transmittance was measured taking into consideration reflection losses at the end faces. The length-independent spectral attenuation can then be calculated from the transmittance and the length of the light guide, and this is given on the chart in dB/km, from which the specification dB/m with 1000 dB/km=1 dB/m, which is relatively common for such fibre optic cables, can be derived.

It is particularly advantageous here that in particular the spectral attenuation 5 of the glass fibre 7 according to the invention in the near-infrared region (NIR), e.g. at 800 nm, wavelength 6, is lower than for a conventional glass fibre 8, as was for example described at the outset. In the example shown, this is 200 dB/km or 0.2 dB/m compared to approx. 350 dB/km or 0.35 dB/m in the conventional glass fibre. This is particularly advantageous in spectroscopic analysis in uses in medical technology, for example in endoscopes, since in particular tissue analyses in the NIR region enable an improved signal-to-noise ratio and thus e.g. imaging contrast can be increased in particular. In addition, any fluctuations in different melts have proven to be less pronounced at this wavelength range than in conventional glasses, which can in particular be ascribed to the composition according to the invention.

On the other hand, however, the first line chart 4 also shows that what is referred to as the UV edge or blue edge for the above-described glass fibre 8 according to the invention is shifted to considerably higher wavelengths than the conventional glass fibre 7; in other words, the spectral attenuation 5 in the blue wavelength region between 400 nm and 500 nm is considerably higher than in the conventional glass fibre 7. As described above, the position of the UV edge or blue edge can be adjusted by the Y₂O₃ or Gd₂O₃ content, or the combination thereof.

Somewhat higher spectral attenuation 5 in the blue region is then in particular not disadvantageous if such glass fibres are used in particular in endoscopic devices. Here, the typical usage lengths are at most 1 m to 2 m, and therefore the “yellow shift”, i.e. a colour shift towards yellow, is not particularly pronounced, which is in particular non-critical for single-use endoscopes which have typical usage lengths of <1 m. Advantageously, the slightly increased attenuation in the blue region can even be utilized if the light guide element, for example a glass fibre, is coupled to a light source which emits more strongly in the blue region, and/or if it is intended to examine tissue which is sensitive to blue light and thus reacts to higher-energy components of the spectrum.

FIG. 3 shows, in a second line chart 9, the results for determining the angular aperture of the glass fibres. To this end, a sensor is customarily used to measure the intensity 10 of the light radiated out by the light guide depending on the angle 11 relative to the radiation axis of the light guide, as is shown for a conventional glass fibre 7 and for the glass fibre 8 according to the invention, in accordance with the glass fibre described above and shown in FIG. 1. What is referred to as the 2α angular aperture then results, according to its definition, from the angles 11 at which the intensity 10 has dropped to 50% of the maximum value at 0°.

As shown in FIG. 3, the two glass fibres 7, 8 have a virtually identical 2α angular aperture=2×approx. 60°=120°, corresponding to a numerical aperture of NA=0.86, where NA=sin⁻¹(α), and therefore both glass fibres can be referred to as wide-angle glass fibres, such as can advantageously be used in endoscopic applications in order to be able to illuminate the field of view of such cameras without shadows, according to the diagonal angular aperture of camera chips.

Further examinations of the glass fibre 8 according to the invention compared to the conventional glass fibre 7, both of which are wide-angle fibres and have a fibre diameter of 70 μm, relate to the level of strength. To this end, 30 glass fibre samples each, of the same length, were tensioned in a tensile testing machine, and the strain at break of the fibres was measured. Accordingly, virtually identical tensile strengths around 1000 MPa strain at break were measured for both glass fibres 7 and 8, with the statistical fluctuation ranges overlapping to such an extent that it can be assumed they both have the same level of tensile strength.

### REFERENCE SIGNS

1 Glass article, light guide element
2 Core glass
3 Cladding glass
4 First Line chart
5 Spectral attenuation
6 Wavelength
7 Conventional glass fibre
8 Light guide element, glass fibre, according to the invention
9 Second Line chart
10 Intensity
11 Angle

What is claimed is:

1. Glass comprising:
SiO₂ and at least one of two components Gd₂O₃ and Y₂O₃, a ratio of a sum of the proportions by weight of Gd₂O₃ and Y₂O₃ to the proportion by weight of SiO₂ is at least 0.01, wherein a proportion of Ta₂O₅ is at most 10 wt %, wherein a proportion of ZrO₂ is at least 0.1 wt %, wherein the ratio of the proportion by weight of B₂O₃ to the proportion by weight of SiO₂ is at most 0.50.

2. The glass as recited in claim 1 wherein the sum of the proportions by weight of Gd₂O₃ and Y₂O₃ is at least 0.2 wt %.

3. The glass as recited in claim 1 wherein a sum of the proportions by weight of BaO and La₂O₃ is at least 20 wt %.

4. The glass as recited in claim 1 wherein the following components are in the stated proportions (by weight):

| Component | Min (wt %) | Max (wt %) |
|---|---|---|
| SiO₂ | 10 | 55 |
| B₂O₃ | 0 | 25 |
| CaO | 0 | 5.0 |
| BaO | 0 | 50 |
| SrO | 0 | 5.0 |
| ZnO | 0 | 30 |
| La₂O₃ | 0 | 70 |
| Gd₂O₃ | 0 | 15 |
| Y₂O₃ | 0 | 15 |
| ZrO₂ | 0.1 | 10 |
| Ta₂O₅ | 0 | 10 |
| Nb₂O₅ | 0 | 10 |
| Σ R₂O | 0 | 10 |

5. The glass as recited in claim 1 wherein the proportion of Y₂O₃ and Gd₂O₃ is in each case at least 0.1 wt %, preferably in each case at least 0.2 wt %.

6. The glass as recited in claim 1 wherein the proportion by weight of Y₂O₃ is greater than the proportion by weight of Nb₂O₅.

7. The glass as recited in claim 1 wherein the proportion of LaₛO₃ is at least 15 wt %.

8. The glass as recited in claim 7 wherein the proportion of La₂O₃ is at most 40 wt %.

9. The glass as recited in claim 1 wherein the ratio of the proportion by weight of La₂O₃ to the sum of the proportions by weight of Gd₂O₃ and Y₂O₃ is at most 10.

10. The glass as recited in claim 1 wherein the sum of the proportions of La₂O₃, Gd₂O₃ and Y₂O₃ is at least 10 wt %.

11. The glass as recited in claim 1 wherein a lower devitrification point is at least 650° C. when the glass is thermally treated for a retention time of 5 minutes in a gradient furnace with rising temperature control.

12. The glass as recited in claim 1 wherein a difference between a lower devitrification point and the softening point is at least 50 K.

13. The glass as recited in claim 1 wherein the proportion of ZrO₂ is less than 10 wt %.

14. A light guide element comprising the glass as recited in claim 1 wherein the refractive index n_{d} of the glass is in a range from 1.65 to 1.80.

15. The light guide element comprising the glass as recited in claim 1 as core glass and a cladding glass surrounding the core glass.

16. The light guide element as recited in claim 15 wherein the cladding glass consists of a borosilicate glass.

17. The light guide element as recited in claim 15 wherein the cladding glass includes the following components of compositions B1 or B2

| Composition | B1 | B2 |
|---|---|---|
| SiO₂ | 60-75 | 60-75 |
| B₂O₃ | 7-25 | 0.5-15 |
| Na₂O | 0-8 | 1-15 |
| K₂O | 0-8 | 0-15 |
| Al₂O₃ | 5-17 | 3-10 |

18. The light guide element as recited in claim 14 having a numerical aperture of at least 0.82.

19. The light guide element as recited in claim 14 having a spectral attenuation in the near IR range at a wavelength of 800 nm of at most 0.3 dB/m.

20. A method for producing the glass as recited in claim 1, the method comprising the following steps:
melting glass raw materials to obtain a glass melt,
cooling a glass or glass article obtained from the glass melt,
processing the glass melt.

21. A method comprising employing the glass as recited in claim 1 as core glass in a light or image guide.

22. A method comprising employing the light guide element as recited in claim 14 in endoscopic applications, in projection apparatuses, in optical data transmission technology, automotive applications, laser technology or disinfection applications.
